(19) United States
(12) Patent Application Publication
(10) Pub. No.: US 2012/0150103 A1
(43) Pub. Date: Jun. 14, 2012

(54) TISSUE ASPIRATION INSTRUMENT EMPLOYING TWIN IRRIGATING-TYPE ELECTRO-CAUTERIZING CANNULA ASSEMBLY

(76) Inventor: Robert L. Cucin, West Palm Beach, FL (US)

(21) Appl. No.: 13/314,396

(22) Filed: Dec. 8, 2011

Related U.S. Application Data
(63) Continuation of application No. 12/813,067, filed on Jun. 10, 2010.

(51) Int. Cl. A61M 1/00; A61M 3/02

(52) U.S. Cl. 604/30; 604/35

(57) ABSTRACT

An irrigating twin-cannula aspiration system including a hand-supportable tissue aspiration instrument, having a twin cannula assembly, wherein an inner cannula having an aspiration aperture is mechanically reciprocated within a stationary outer cannula having an elongated aperture along which the aspiration aperture reciprocates, while the inner cannula is operably affixed to a vacuum source, and a controller controls synchronized pulsed tumescent infusion about the aspiration aperture during system operation.

### RELATED CASES

This application is a Continuation of copending application Ser. No. 12/813,067 filed Jun. 10, 2010, said Application is owned by Rocin Laboratories, Inc., and incorporated herein by reference in its entirety.

### BACKGROUND OF INVENTION

1. Field of Invention

The present invention relates generally to new and improved hand-supportable power-assisted instruments for performing liposuction and other tissue aspiration operations, in a mechanically assisted manner using powered expedients, and improved methods of operating and using the same.

2. Brief Description of the State of the Knowledge in the Art

Suction lipectomy, commonly known as liposuction or lipoxheresis, is a well known surgical procedure used for sculpturing or contouring the human body to increase the attractiveness of its form. In general, the procedure involves the use of a special type of curet known as a cannula, which is operably connected to a vacuum source. The cannula is inserted within a region of fatty tissue where removal thereof is desired, and the vacuum source suctions the fatty tissue through the suction aperture in the cannula and carries the aspirated fat away. Removal of fat cells by liposuction creates a desired contour that will retain its form.

Presently, there are two widely accepted techniques of liposuction and each may be practiced using a conventional liposuction cannula. The first and most common method proposed by Yves-Gerard Illouz and described in the paper “Illouz's Technique of Body Contouring by Lipolysis” in Vol. 3, No. 3, July 1984 of Clinics in Plastic Surgery, involves making regular tunnels at a depth of at least 1 centimeter under the skin. According to this method, one or two insertions are made, with radial excursions of the cannula into the fatty tissue of the patient. The result is a multitude of concomitant sinuses formed below the subcutaneous fatty tissue, leaving intact as far as possible the connections between the skin and underlying tissue, thereby retaining the blood vessels, the lymphatics and the nerve endings. The second method is the original liposuction procedure proposed by U. K. Kesselring, described in “Body Contouring with Suction Lipectomy,” in Vol. 11, No. 3, July 1984, Clinics in Plastic Surgery. According to the technique, an entire layer of regular, deep fat is removed by aspiration through the cannula, leaving a smooth, deep surface of the residual panniculus. The space thus created is then compressed, optimally followed by skin retraction.

Both of these prior art liposuction techniques require that the surgeon push and pull the entire cannula back and forth almost twenty times for each insertion made. Typically, twenty to thirty tunnels are made. This is necessary to ensure even removal of fat in the targeted region. During this procedure, the surgeon typically massages the flesh in the area of the aperture in the cannula, while at the same time thrusting the rod in and out of the tunnel. Due to the trauma involved during the procedure, the patient's skin turns black and blue for several weeks. Due to the physically exacting nature of the procedure, the surgeon typically comes out of an operating room extremely tired and suffers from muscular fatigue which prevents him from performing, for some time thereafter, delicate operations involved in ordinary plastic surgery.

Recently, the use of a “guided cannula” has been proposed by R. de la Plaza, et al., described in “The Rationalization of Liposuction Toward a Safer and More Accurate Technique,” published in vol. 13, Aesthetic Plastic Surgery, 1989. According to the technique, a cannula is used in conjunction with an outer guide sheath through which the cannula can slidably pass while held in place by the handle portion of the guide sheath. Once the cannula and its sheath have been introduced into the fatty tissue, the sheath guide remains in the tunnel and guides successive introductions of the cannula, keeping it in the same tunnel. While the use of this liposuction technique offers some advantages over the conventional unguided liposuction cannulas, the guided cannula nevertheless suffers from several significant shortcomings and drawbacks. In particular, the guided cannula requires manually thrusting the cannula through the guide sleeve repeatedly for each tunnel. Although this is a less physically demanding procedure, the surgeon must thrust the cannula even more times through each tunnel to achieve the desired effect and hence is still easily fatigued and prevented from performing, for some time thereafter, delicate operations involved in ordinary plastic surgery.

In an attempt to solve the above-described problem, U.S. Pat. Nos. 4,735,605 and 4,775,365 and 4,792,327 to Swartz disclose an assisted lipectomy cannula having an aspiration aperture which effectively travels along a portion of the length of the cannula, thereby obviating the necessity of the surgeon to repeatedly push the cannula in and out of the patient's subcutaneous tissue where fatty tissue is to be removed. While this assisted lipectomy cannula can operate on either air or electric power, it nevertheless suffers from several significant shortcomings and drawbacks. In particular, the device requires an outer tube with an elongated slot and an inner tube having a spiral slot which must be rotated inside the outer tube to effectuate a traveling aspiration aperture. In addition to the device's overall construction posing difficulties in assembly, cleaning and sterilization, use with a variety of cannulas and highly effective fat aspiration does not appear possible.

In U.S. Pat. No. 5,112,302 to Cucin, Applicant discloses a powered liposuction instrument which offers significant improvements over the instruments disclosed in US Letters Patents above. However, the powered liposuction instrument designs taught in U.S. Pat. No. 5,112,302 are not without shortcomings and drawbacks. In particular, these liposuction instrument designs employ a single cannula which is designed to reciprocate relative to the instrument housing by relatively large amounts (e.g. 1-10 centimeters). When using instruments of this prior art design, it is possible that such large scale movements of the cannula can accidently rupture tissue walls within the patient, causing complications which are best avoided by practicing surgeons at all costs.

Also, while U.S. Pat. Nos. 6,872,701 and 7,381,206 to Cucin disclose liposuction instruments having dual cannula assemblies, and electro-cauterizing cannula assemblies, these instruments too have suffered from a number of shortcoming and drawbacks which have necessitated improvements in the art.

In particular, loading of the cannula(s) into the hand-supportable housing of prior art single and twin cannula type liposuction instruments has not been as simple as possible.

Also, the cleaning of prior art liposuction instruments after each use in an autoclave has also been very difficult because of the incompatibility, of water with electrical componentry, the heat sensitivity of pneumatic seals and/or electronic components, and the softening and heat set of autoclaved materials into the tightly coiled positions required for insertion into standard-sized steam autoclaves.

Establishing a secure connection of the aspiration tube to the cannula assembly has also been difficult due to relative motion in the cannula in such powered instruments, sometimes causing the aspiration tube to disconnect from the instrument during operation.

Also, due to conventional design and construction techniques used in prior art liposuction instruments, such powered liposuction instruments have been every expensive to manufacture and maintain, making reliable operation difficult to ensure, and putting such instruments out of the hands of most surgeons in today's challenging economy.

Accordingly, there is a great need in the art for new and improved mechanically-assisted lipectomy instruments which overcome the shortcomings and drawbacks of prior art lipectomy apparatus.

### OBJECTIVES AND SUMMARY OF THE PRESENT INVENTION

Thus, it is a primary object of the present invention to provide an improved method and apparatus for performing liposuction which assists the surgeon in the removal of fat and other subcutaneous tissue (such as but not restricted to gynecomastia) from surrounding tissue, with increased safety and without promoting physical fatigue.

Another object of the present invention to provide a tissue aspiration instrumentation system which comprises a hand-supportable tissue aspiration instrument having a hand-supportable housing with a stationary tubing connector provided at the rear of the housing and receiving a length of flexible tubing connected to a vacuum source, and including a single cannula assembly coupled to a cannula drive mechanism disposed within the hand-supportable housing and powered by an external power source (e.g. electrical power signals, pressurized air-streams, etc) so as to periodically exert forces on the cannula base portion along the longitudinal axis of the cannula assembly (i.e. coaxially exerted on the cannula base portion) and cause the hollow cannula base portion to reciprocate within the cylindrical (cannula base portion) guide tube, while tissue is being aspirated along the cannula lumen, through the lumen formed in the cannula base portion, through the cylindrical guide tube and through the stationary tubing connector, along the flexible tubing towards the vacuum source.

Another object of the present invention is to provide a tissue-aspiration instrumentation system which comprises a hand-supportable tissue aspiration instrument and a single-type cannula assembly, wherein the hand-supportable tissue aspiration instrument includes (i) a hand-supportable housing having (i) a front portion and a rear portion aligned along a longitudinal axis, (ii) an interior volume and a cylindrical guide tube mounted within the interior volume, (iii) a cannula drive mechanism disposed adjacent the cylindrical guide tube, and (iv) a stationary tubing connector coaxially mounted to the rear portion of the hand-supportable housing along the longitudinal axis, connected to the cylindrical guide tube, and having an exterior connector portion permitting a section of flexible aspiration tubing to be connected at its first end to the exterior connector portion, and where the second end of the section of flexible tubing is connected to a vacuum source.

Another object of the present invention is to provide a tissue-aspiration instrumentation system which comprises a hand-supportable tissue aspiration instrument and a twin-type cannula assembly.

An even further object of the present invention is to provide such a tissue aspiration instrument which can be driven by pressurized air or electricity.

A further object of the present invention is to provide such a liposuction instrument, in which the cannula assembly is disposable.

An even further object of the present invention is to provide an improved method of performing liposuction, in which one of the cannulas of the cannula assembly is automatically reciprocated back and forth relative to the hand-holdable housing, to permit increased control over the area of subcutaneous tissue where fatty and other soft tissue is to be aspirated.

Another object of the present invention is to provide a power-assisted liposuction instrument, with a means along the cannula assembly to effect hemostasis during liposuction procedures and the like, using RF-based electro cauterization.

Another object of the present invention is to provide an air-powered tissue-aspiration (e.g., liposuction) instrument system, wherein the powered liposuction instrument has an inner cannula that is automatically reciprocated within a stationary outer cannula by electronically controlling the flow of pressurized air streams within a dual-port pressurized air cylinder supported within the hand-supportable housing of the instrument.

Another object of the present invention is to provide such an air-powered liposuction instrument system, wherein digital electronic control signals are generated within an instrument controller unit and these control signals are used to generate a pair of pressurized air streams within the instrument controller which are then supplied to opposite ends of the dual-port pressurized air cylinder within the powered liposuction instrument.

Another object of the present invention is to provide such an air-powered liposuction instrument system, wherein the rear end of the powered liposuction instrument has a pressurized air-power supply-line connector, and an electrical control signal connector.

Another object of the present invention is to provide such an air-powered liposuction instrument system, wherein the hollow inner cannula base portion of cannula assembly inserts into a front accessible port in the hand-supportable housing, while the aspiration tubing is connected to the stationary tube connector provided at the rear portion of the hand-supportable housing.

Another object of the present invention is to provide such an air-powered tissue-aspiration instrument system, wherein an intelligent instrument controller is used to supply air-power to the inner cannula reciprocation mechanism within the hand-supportable instrument, while communicating control signals between the instrument and its intelligent controller.

Another object of the present invention is to provide such an tissue-aspiration instrument system with an alternative electro-cauterizing dual cannula assembly, wherein a stream of irrigation fluid is automatically pumped from the base portion of the outer cannula to the distal portion thereof, along a micro-sized fluid conduit formed along the surface walls of the outer cannula, and released into the interior distal portion of the outer cannula through a small opening formed therein, for infiltration and irrigation of tissue during aspiration in order to facilitate pump action.

These and other Objects of the present invention will become apparent hereinafter.

### BRIEF DESCRIPTION OF THE DRAWINGS

For a fuller understanding of the objects of the present invention, reference is made to the detailed description of the illustrative embodiments which are to be taken in connection with the accompanying drawings, wherein;

FIG. 1A is a schematic representation of a first generalized embodiment of the tissue aspiration instrumentation system of the present invention, comprising a hand-supportable tissue aspiration instrument having a hand-supportable housing with a stationary tubing connector provided at the rear of the housing and receiving a length of flexible tubing connected to a vacuum source, and including a single cannula assembly coupled to a cannula drive mechanism disposed within the hand-supportable housing and powered by an external power source (e.g. electrical power signals, pressurized air-streams, etc);

FIG. 1B is a cross-sectional view of the hand-supportable tissue aspiration instrument shown in FIG. 1A, showing its single cannula being reciprocated relative to the hand-supportable housing, as its hollow cannula base portion is reciprocated within the cylindrical (cannula base portion) guide tube, and tissue is aspirated along the cannula lumen, through the lumen formed in the cannula base portion, through the cylindrical guide tube and through the stationary tubing connector, along the flexible tubing towards the vacuum source;

FIG. 1C is a schematic representation of a second generalized embodiment of the tissue aspiration instrumentation system of the present invention, comprising a hand-supportable tissue aspiration instrument having a hand-supportable housing with a stationary tubing connector provided at the rear of the housing and receiving a length of flexible tubing connected to a vacuum source, and including a twin-cannula assembly having an inner cannula coupled to a cannula drive mechanism disposed within the hand-supportable housing and powered by an external power source (e.g. electrical power signals, pressurized air-streams, etc), while its stationary outer cannula is releasably connected to the front portion of the hand-supportable housing;

FIG. 1D is a cross-sectional view of the hand-supportable tissue aspiration instrument shown in FIG. 1C, showing its inner cannula being reciprocated relative to the hand-supportable housing, as its hollow inner cannula base portion is reciprocated within the cylindrical (cannula base portion) guide tube, and tissue is aspirated along the inner annula lumen, through the lumen formed in the inner cannula base portion, through the cylindrical guide tube, through the stationary tubing connector, and along the flexible tubing towards the vacuum source;

FIG. 2A is a perspective view of a first illustrative embodiment of the tissue aspiration instrumentation system of the present invention, comprising a hand-supportable tissue aspiration instrument having (i) a hand-supportable housing with a stationary tubing connector provided at the rear of the housing and receiving a length of flexible tubing connected to a vacuum source, and (ii) a single-cannula assembly having an inner cannula coupled to an electromagnetic-based cannula drive mechanism disposed within the hand-supportable housing and powered by an AC electrical signal power source, while its stationary outer cannula is releasably connected to the front portion of the hand-supportable housing;

FIG. 2B is an elevated side view of the first illustrative embodiment of the tissue aspiration instrumentation system of the present invention shown in FIG. 2A;

FIG. 2C is a plan view of the tissue aspiration instrumentation system of the present invention shown in FIGS. 2A and 2B;

FIG. 2D is an elevated rear view of the tissue aspiration instrumentation system of the present invention shown in FIGS. 2A, 2B and 2C;

FIG. 3A is a cross-sectional view of the hand-supportable tissue aspiration instrument shown in FIG. 2B;

FIG. 3B is an enlarged view of the cross-sectional view of the hand-supportable tissue aspiration instrument of FIGS. 2A and 2B, showing the cylindrical guide tube mounted within the hand-supportable housing, the cannula base portion carrying a permanent magnetic ring between a set of fluid seals that slidably support the cannula base portion within the cylindrical guide tube, the cannula coupled to the cannula base portion by way of a leur-lock fitting, and the lumen extending within the cannula and its base portion being in fluid communication with the stationary tubing connector, by way of the interior volume of the cylindrical guide tube between the cannula base portion and the stationary tubing connector;

FIG. 4A is a first exploded view of the hand-supportable tissue aspiration instrument of FIGS. 2A and 2B, showing its primary components;

FIG. 4B is a second exploded view of the hand-supportable tissue aspiration instrument of FIGS. 2A and 2B, showing a first step in the assembly of the hand-supportable tissue aspiration instrument;

FIG. 4C is a third exploded view of the hand-supportable tissue aspiration instrument of FIGS. 2A and 2B, showing a second step in the hand-supportable tissue aspiration instrument;

FIG. 4D is a fourth exploded view of the hand-supportable tissue aspiration instrument of FIGS. 2A and 2B, showing a third step in the hand-supportable tissue aspiration instrument;

FIG. 4E is a fifth exploded view of the hand-supportable tissue aspiration instrument of FIGS. 2A and 2B, showing a fourth step in the hand-supportable tissue aspiration instrument;

FIG. 4F is a sixth exploded view of the hand-supportable tissue aspiration instrument of FIGS. 2A and 2B, showing a fifth step in the hand-supportable tissue aspiration instrument;

FIG. 4G is a seventh exploded view of the hand-supportable tissue aspiration instrument of FIGS. 2A and 2B, showing a sixth step in the hand-supportable tissue aspiration instrument;

FIG. 4H is an eighth exploded view of the hand-supportable tissue aspiration instrument of FIGS. 2A and 2B, showing a seventh step in the hand-supportable tissue aspiration instrument;

FIG. 4I is a ninth exploded view of the hand-supportable tissue aspiration instrument of FIGS. 2A and 2B, showing an eighth step in the hand-supportable tissue aspiration instrument;

FIG. 4J is a tenth exploded view of the hand-supportable tissue aspiration instrument of FIGS. 2A and 2B, showing a ninth step in the hand-supportable tissue aspiration instrument;

FIG. 4K is a perspective view of the hand-supportable tissue aspiration instrument of FIGS. 2A and 2B, showing the hand-supportable tissue aspiration instrument fully assembled;

FIG. 5A is a perspective view of the back housing plate;

FIG. 5B is a perspective view of the cylindrical guide tube supporting its first and second electromagnetic coils;

FIG. 5C is an elevated side view of the cylindrical guide tube supporting its first and second electromagnetic coils;

FIG. 5D is a perspective partially-cutaway view showing the connection of the two electromagnetic coils to the contact plug employed in the hand-supportable tissue aspiration instrument of the present invention illustrated in FIG. 3B;

FIG. 5E is schematic diagram of a two coil push-pull type of circuit for enabling the cannula drive mechanism employed in the hand-supportable tissue aspiration instrument of the present invention illustrated in FIG. 3B;

FIG. 6A is a sectional-view of a second embodiment of the hand-supportable tissue aspiration instrument of FIGS. 2A and 2B, showing a cylindrical (cannula base portion) guide tube supporting three electromagnetic coils used to realize the cannula drive mechanism employed in the tissue aspiration instrument;

FIG. 6B is schematic diagram of a three coil push-pull type of circuit for enabling the cannula drive mechanism employed in the second embodiment of the hand-supportable tissue aspiration instrument of the present invention illustrated in FIG. 6A;

FIG. 6C is a sectional-view of a third embodiment of the hand-supportable tissue aspiration instrumentation system of the present invention, showing a cylindrical (cannula base portion) guide tube supporting three electromagnetic coils used to realize the cannula drive mechanism employed in the tissue aspiration instrument;

FIG. 6D is schematic diagram of a three coil push-pull type of circuit for enabling the cannula drive mechanism employed in the third embodiment of the hand-supportable tissue aspiration instrument of the present invention illustrated in FIG. 6C;

FIG. 7A is a perspective view of a fourth illustrative embodiment of the tissue aspiration instrumentation system of the present invention, comprising a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a single-cannula assembly having an inner cannula coupled to an pneumatically-powered cannula drive mechanism disposed within the hand-supportable housing and powered by a source of pressurized air or other gas, while its stationary outer cannula is releasably connected to the front portion of the hand-supportable housing;

FIG. 7B is an elevated side view of the air-powered tissue aspiration instrument shown in FIG. 7A, wherein a single-button quick connect plug and associated multi-core cable assembly is provided on the rear portion of the hand-supportable housing, for supporting two gas lines and three electric wires between the instrument and its controller in a single bundle;

FIG. 7C is a partially exploded diagram of the fourth illustrative embodiment of the tissue aspiration instrumentation system of the present invention, showing its hand-supporting housing, in which its cylindrical (cannula base portion) guide tube and air-powered driven mechanism are installed, while its cannula base portion, cannula and cannula lock nut are shown disassembled outside of the hand-supportable housing;

FIG. 8A is a cross-sectional view of the hand-supportable tissue aspiration instrumentation system of FIG. 8A, shown configured with its aspiration source, its controller and pneumatic power source, and multi-core cable assembly;

FIG. 8B is a schematic representation of the controller (and air-power supply) console depicted in hybrid schematic diagram of FIG. 8A, illustrating the front and rear Hall-effect cannula base position sensors installed within the hand-supportable housing of the instrument, the LCD panel, communication ports, LED indicators, and panel membrane switches supported on the controller console housing, as well as the ADC, digital signal processor (DSP) and DAC and proportional valve contained within the controller console housing and supplying gas tubes (via the multi-code cable assembly), and a supply of pressurized gas supplied to the controller housing, for driving the cannula drive mechanism of this embodiment of the present invention;

FIG. 9A is a perspective view of a fifth illustrative embodiment of the tissue aspiration instrumentation system of the present invention, comprising a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a twin-cannula assembly having an inner cannula coupled to an pneumatically-powered cannula drive mechanism disposed within the hand-supportable housing and powered by a source of pressurized air or other gas, while its stationary outer cannula is releasably connected to the front portion of the hand-supportable housing;

FIG. 9B is an elevated side view of the air-powered tissue aspiration instrument shown in FIG. 9A, wherein a single-button quick connect plug and associated multi-core cable assembly is provided on the rear portion of the hand-supportable housing, for supporting two gas lines and three electric wires between the instrument and its controller in a single bundle;

FIG. 10A is a perspective view of a sixth illustrative embodiment of the tissue aspiration instrumentation system of the present invention, comprising a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a single-cannula assembly having an inner cannula coupled to an linear-actuator powered cannula drive mechanism disposed within the hand-supportable housing and powered by a source of electrical power, while its stationary outer cannula is releasably connected to the front portion of the hand-supportable housing;

FIG. 10B is an elevated side view of the air-powered tissue aspiration instrument shown in FIG. 10A, wherein the linear actuator (i.e. linear motor) is mounted within the hand-supportable housing, outside of the cylindrical (cannula base portion) guide tube, and driven by electrical power signals supplied through a quick-release type connector, connecting a flexible electrical signal cable between the instrument and its controller console;

FIG. 11A is a perspective view of a seventh illustrative embodiment of the tissue aspiration instrumentation system of the present invention, comprising a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a twin tumescent-type cannula assembly having an inner cannula coupled to an pneumatically-powered cannula drive mechanism disposed within the hand-supportable housing and powered by a source of pressurized air or other gas, while its stationary outer cannula is releasably connected to the front portion of the hand-supportable housing;

FIG. 11B is an elevated side view of the air-powered tissue aspiration instrument shown in FIG. 11A, shown configured with its aspiration source, controller, pneumatic power source, and multi-core cable assembly, and the tumescent outer cannula infusion port connected to an infusion pump that is daisy chained to the controller for synchronized pulse control, and operational to synchronize the release of irrigation fluid (i.e. infusion) with inner cannula motion;

FIG. 11C is a cross-sectional view of the tissue aspiration instrument shown in FIG. 11B;

FIG. 12A is an elevated side view of the air-powered tissue aspiration instrument shown in FIGS. 11A and 11B, wherein the tumescent outer cannula is shown attached to chamber screw cap, allowing the base portion of the outer cannula to be screwed on same threads of the chamber screw cap, and preferably will be a larger size luer lock style fitting so as to assure registration of slot and hole, because the use of tumescent cannula requires that the reciprocally driven inner cannula base portion be “keyed” so as not to rotate within and maintain constant alignment with the slotted outer cannula;

FIG. 12B is a perspective view of the tumescent outer cannula shown in FIG. 12A, illustrating a tab for screwing on (i.e. fastening) the outer cannula over the inner cannula, and aligning the outer cannula with respective the inner cannula, realizable by threads applied to the inside or a large luer-lock fitting on the chamber screw cap and base portion of outer cannula; and

FIG. 12C is an end view of the base portion of the outer cannula shown in FIG. 12B; and

FIG. 12D is a perspective partially cut-away view of the tumescent cannula tip port in of the tumescent outer cannula employed in the instrument shown in FIGS. 11A and 11B.

### DETAILED DESCRIPTION OF THE ILLUSTRATED EMBODIMENTS

Referring to the figures in the accompanying Drawings, the various illustrative embodiments of the wireless mobile advertising network and components of the present invention will be described in great detail, wherein like elements will be indicated using like reference numerals.

First Generalized Embodiment of the Tissue Aspiration Instrumentation System of the Present Invention, Provided with a Single Cannula Assembly

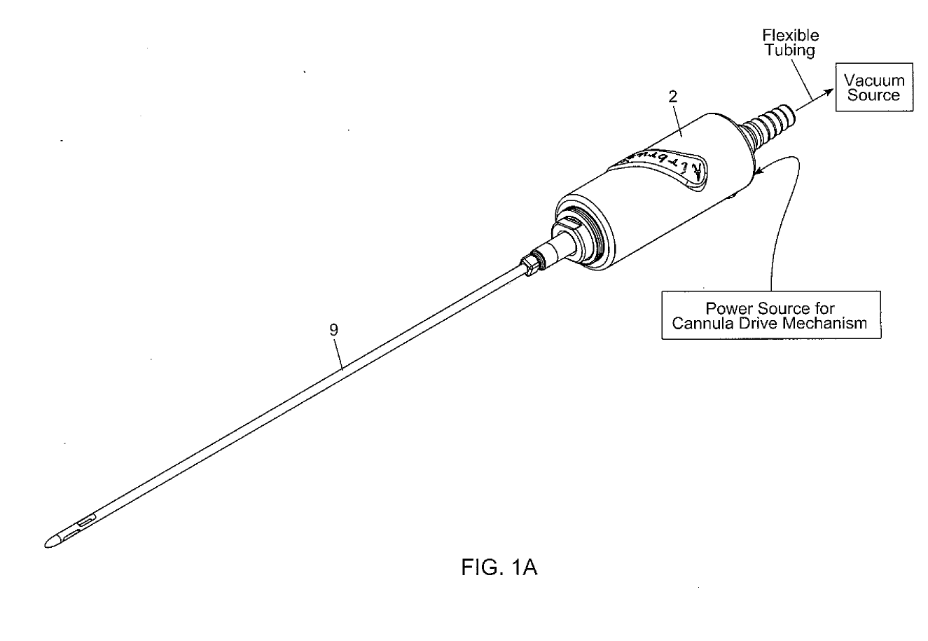

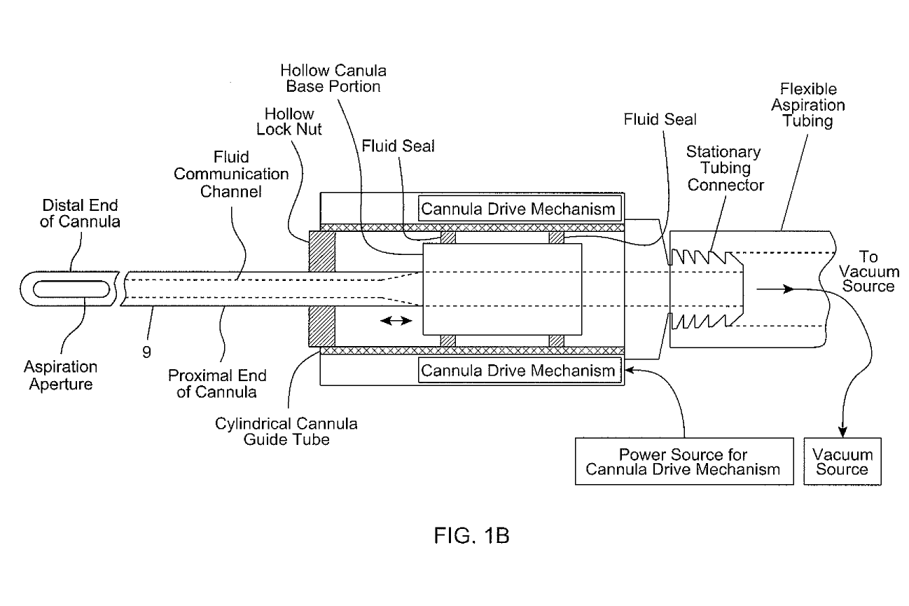

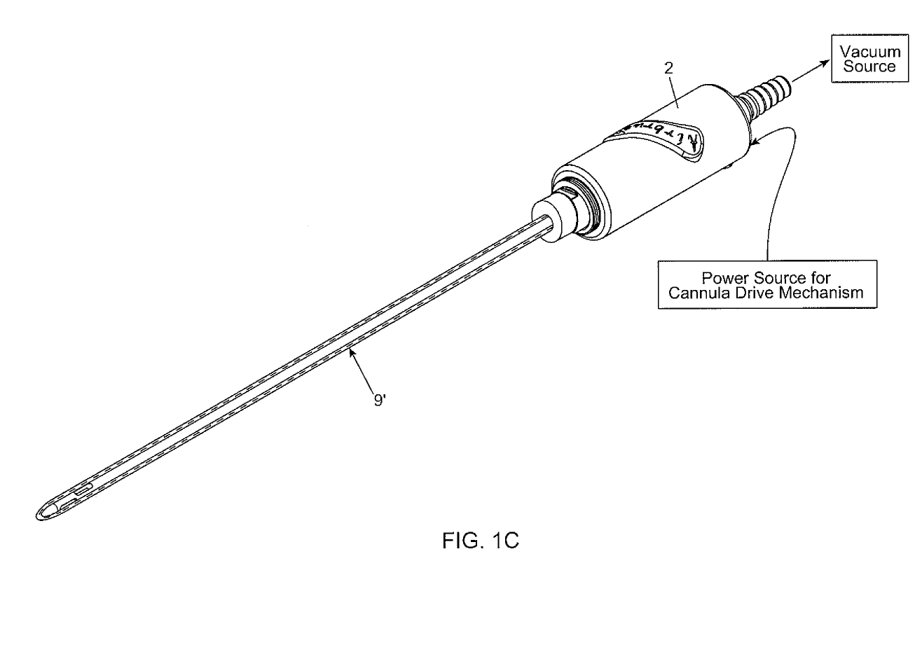

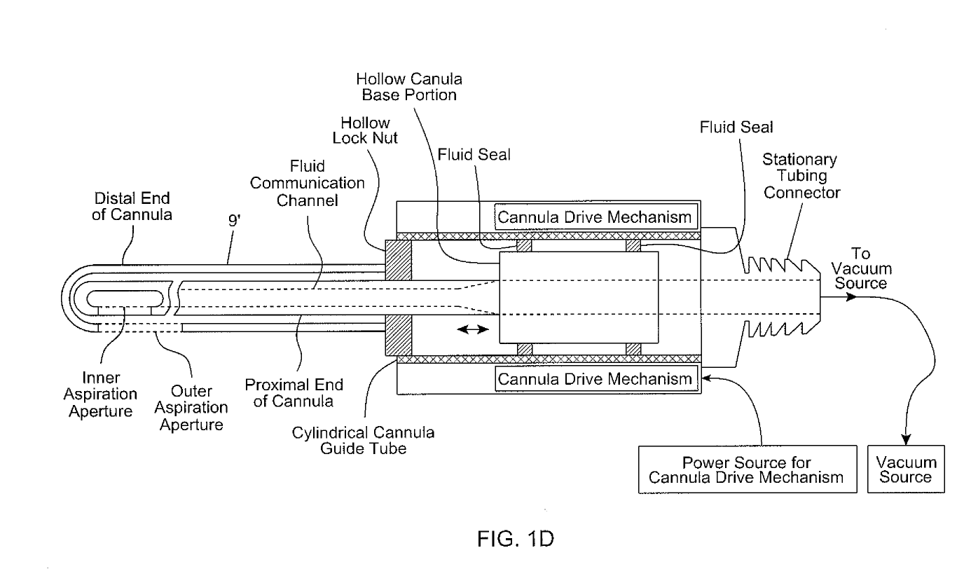

With reference to FIGS. 1A and 1B, the first generalized embodiment of the coaxial-powered tissue aspiration instrument of the present invention will be described.

In general, the first generalized embodiment of the tissue aspiration instrumentation system of the present invention comprises a hand-supportable tissue aspiration instrument with a single cannula assembly. The instrument has a hand-supportable housing with a stationary tubing connector provided at the rear of the housing and receiving a length of flexible tubing connected to a vacuum source. The single cannula assembly is coupled to a cannula drive mechanism disposed within the hand-supportable housing and powered by an external power source (e.g. electrical power signals, pressurized air-streams, etc) so as to exert coaxially-directed forces on the cannula base portion along the longitudinal axis of the cannula assembly (i.e. coaxially exerted on the cannula base portion) and cause the hollow cannula base portion to reciprocate within the cylindrical (cannula base portion) guide tube, while tissue is being aspirated along the cannula lumen, through the lumen formed in the cannula base portion, through the cylindrical guide tube and through the stationary tubing connector, along the flexible tubing towards the vacuum source. This arrangement effectuates periodic displacement of the general location of aspiration along the cannula assembly through the reciprocating movement of cannula while the aspiration tubing is maintained substantially stationary relative to the hand-supportable housing during operation of the tissue aspiration instrument, thereby providing smooth operation, substantially free of any push/pulling action on the flexible aspiration tubing connected between the instrument and the vacuum source, during surgical operations.

As illustrated in FIGS. 1A and 1B, first generalized embodiment of the tissue-aspiration instrument of the present invention comprises: (i) a hand-supportable housing having (i) a front portion and a rear portion aligned along a longitudinal axis, (ii) an interior volume and a cylindrical guide tube mounted within the interior volume, (iii) a cannula drive mechanism disposed adjacent the cylindrical guide tube, and (iv) a stationary tubing connector coaxially mounted to the rear portion of the hand-supportable housing along the longitudinal axis, connected to the cylindrical guide tube, and having an exterior connector portion permitting a section of flexible aspiration tubing to be connected at its first end to the exterior connector portion, and where the second end of the section of flexible tubing is connected to a vacuum source. The single cannula assembly has a hollow cannula with an outer aspiration aperture extending from the front portion of the hand-supportable housing and having a hollow cannula base portion provided with fluid seals engaging with the inner surface of the cylindrical guide tube.

The function of the cannula drive mechanism is to cause the hollow cannula base portion to reciprocate within the cylindrical guide tube, and thereby reciprocate the aspiration aperture to relative to the hand-supportable housing while tissue is being aspirated through the aspiration aperture and along a fluid communication channel extending from the aspiration aperture, along the hollow cannula and the hollow cannula base portion, and through and along the cylindrical guide tube, and through the stationary tubing connector and into the section of flexible tubing connected to the vacuum source.

As will be described in the illustrative embodiments, there are many ways to realize the components of the instrument system.

One illustrative embodiment, the hollow cannula base portion comprises a tubular structure having a permanent magnet ring mounted about its outer surface and concentric with the longitudinal axis of said hollow inner cannula. Also, the cannula drive mechanism comprises at least one electromagnetic wire coil wound about said cylindrical guide tube, and for generating an electromagnetic force field that is driven by an electrical signal source, and electrically connected to an electrical signal source, for generating an electromagnetic force field which periodically pushes and pulls the permanent magnet ring and thereby causes (i) the hollow cannula base portion to reciprocate within the cylindrical guide tube, (ii) the aspiration aperture to reciprocate relative to the hand-supportable housing.

In another illustrative embodiment, the permanent magnet ring and the at least one electromagnetic coil form a magnetic coupling mechanism between the hollow cannula base portion and the cylindrical guide tube.

The hollow cannula base portion comprises a tubular structure having a permanent magnet ring mounted about its outer surface and concentric with the longitudinal axis of the hollow inner cannula. The cannula drive mechanism comprises a pair of spaced apart electromagnetic wire coils wound about the cylindrical guide tube, and electrically connected to an electrical signal source, for generating an electromagnetic force field which periodically pushes and pulls said permanent magnet ring and thereby causes (i) the hollow cannula base portion to reciprocate within the cylindrical guide tube, (ii) the aspiration aperture to reciprocate relative to the hand-supportable housing.

Alternatively, the cannula drive mechanism can be realized using an pneumatically source of pressurized air or gas, controllably supplied to a coaxially-arranged pneumatically-powered cannula drive mechanism, or linear actuator powered cannula drive mechanism.

In another illustrative embodiment, the permanent magnet ring and the pair of spaced apart electromagnetic coils form a magnetic coupling mechanism between the hollow cannula base portion and the cylindrical guide tube.

The stationary tubing connector comprises a barb-type connector to receiving and gripping the end portion of flexible aspiration tubing.

Each fluid seal comprises an elastomeric or rubber ring that fits tightly against the hollow cannula base portion, and slides along the inner surface of the cylindrical guide tube in a low friction manner.

In yet other embodiments, these elements of invention may be realized in different ways without departing from the scope and spirit of the present invention.

Second Generalized Embodiment of the Tissue Aspiration Instrumentation System of the Present Invention, Provided with a Twin Cannula Assembly

With reference to FIGS. 1C and 1D, the second generalized embodiment of the coaxial-powered tissue aspiration instrument of the present invention will be described.

In general, the second generalized embodiment of the tissue aspiration instrumentation system of the present invention comprises a hand-supportable tissue aspiration instrument having a hand-supportable housing with a stationary tubing connector provided at the rear of the housing and receiving a length of flexible tubing connected to a vacuum source, and including a twin-cannula assembly having an inner cannula coupled to a cannula drive mechanism disposed within the hand-supportable housing and powered by an external power source (e.g. electrical power signals, pressurized air-streams, etc) so as to periodically exert forces on the cannula base portion along the longitudinal axis of the cannula assembly (i.e. coaxially exerted on the cannula base portion) and cause the hollow cannula base portion to reciprocate within the cylindrical (cannula base portion) guide tube, while tissue is being aspirated along the cannula lumen, through the lumen formed in the cannula base portion, through the cylindrical guide tube and through the stationary tubing connector, along the flexible tubing towards the vacuum source, while its stationary outer cannula is releasably connected to the front portion of the hand-supportable housing. This arrangement effectuates periodic displacement of the general location of aspiration along the cannula assembly through the reciprocating movement of inner cannula within the stationary outer cannula, while the aspiration tubing is maintained substantially stationary relative to the hand-supportable housing during operation of the tissue aspiration instrument, thereby providing smooth operation, substantially free of any push/pulling action on the flexible aspiration tubing connected between the instrument and the vacuum source, during surgical operations.

As illustrated in FIGS. 1C and 1D, the second generalized embodiment of the tissue-aspiration instrument of the present invention comprises: a hand-supportable tissue aspiration instrument and a twin-type cannula assembly. The hand-supportable tissue aspiration instrument includes a hand-supportable housing having (i) a front portion and a rear portion aligned along a longitudinal axis, (ii) an interior volume and a cylindrical guide tube mounted within the interior volume, (iii) a cannula drive mechanism disposed adjacent the cylindrical guide tube, and (iv) a stationary tubing connector coaxially mounted to the rear portion of the hand-supportable housing along the longitudinal axis, connected to said cylindrical guide tube, and having an exterior connector portion permitting a section of flexible aspiration tubing to be connected at its first end to the exterior connector portion, and where the second end of the section of flexible tubing is connected to a vacuum source. The twin cannula assembly has a hollow outer cannula with an elongated outer aspiration aperture and having a outer cannula base portion stationarily connected to the front portion of the hand-supportable housing, and a hollow inner cannula with an inner aspiration aperture and disposed within the hollow outer cannula and having an hollow inner cannula base portion provided with fluid seals engaging with the inner surface of the cylindrical guide tube.

The function of the cannula drive mechanism is to cause (i) the hollow inner cannula base portion to reciprocate within the cylindrical guide tube, (ii) the hollow inner cannula to reciprocate within the hollow outer cannula, and (iii) the inner aspiration aperture to reciprocate along the elongated outer aspiration aperture, while tissue is being aspirated through the elongated outer aspiration aperture and through the reciprocating inner aspiration aperture, and along a fluid communication channel extending from the inner aspiration aperture, along the hollow inner cannula and the hollow inner cannula base portion through and along the cylindrical guide tube, and through the stationary tubing connector and into the section of flexible tubing connected to the vacuum source.

As will be described in the illustrative embodiments, there are many ways to realize the components of the instrument system.

One illustrative embodiment, the hollow inner cannula base portion comprises a tubular structure having a permanent magnet ring mounted about its outer surface and concentric with the longitudinal axis of the hollow inner cannula. The cannula drive mechanism comprises at least one electromagnetic wire coil wound about the cylindrical guide tube, and connected to an electrical signal source, for generating an electromagnetic force field which periodically pushes and pulls the permanent magnet ring and thereby causes (i) the hollow inner cannula base portion to reciprocate within the cylindrical guide tube, (ii) the hollow inner cannula to reciprocate within the hollow outer cannula, and (iii) the inner aspiration aperture to reciprocate along the elongated outer aspiration aperture.

In another illustrative embodiment, the permanent magnet ring and at least one electromagnetic coil form a magnetic coupling mechanism between the hollow inner cannula base portion and the cylindrical guide tube.

In another illustrative embodiment, the hollow inner cannula base portion comprises a tubular structure having a permanent magnet ring mounted about its outer surface and concentric with the longitudinal axis of the hollow inner cannula. The cannula drive mechanism comprises a pair of spaced apart electromagnetic wire coils wound about the cylindrical guide tube, and electrically connected to an electrical signal source, for generating an electromagnetic force field which periodically pushes and pulls said permanent magnet ring and thereby causes (i) the hollow inner cannula base portion to reciprocate within the cylindrical guide tube, (ii) the hollow inner cannula to reciprocate within the hollow outer cannula, and (iii) inner aspiration aperture to reciprocate along the elongated outer aspiration aperture.

Alternatively, the cannula drive mechanism can be realized using an pneumatically source of pressurized air or gas, controllably supplied to a coaxially-arranged pneumatically-powered cannula drive mechanism, or linear actuator powered cannula drive mechanism.

In another illustrative embodiment, the permanent magnet ring and the pair of spaced apart electromagnetic coils form a magnetic coupling mechanism between the hollow inner cannula base portion and the cylindrical guide tube.

In the preferred embodiment, the stationary tubing connector comprises a barb-type connector to receiving and gripping the end portion of flexible aspiration tubing.

Also, each fluid seal comprises an elastomeric or rubber ring that fits tightly against the hollow inner cannula base portion, and slides along the inner surface of the cylindrical guide tube in a low friction manner.

In yet other embodiments, these elements of invention may be realized in different ways without departing from the scope and spirit of the present invention.

### First Illustrative Embodiment of the Tissue Aspiration Instrumentation System of the Present Invention

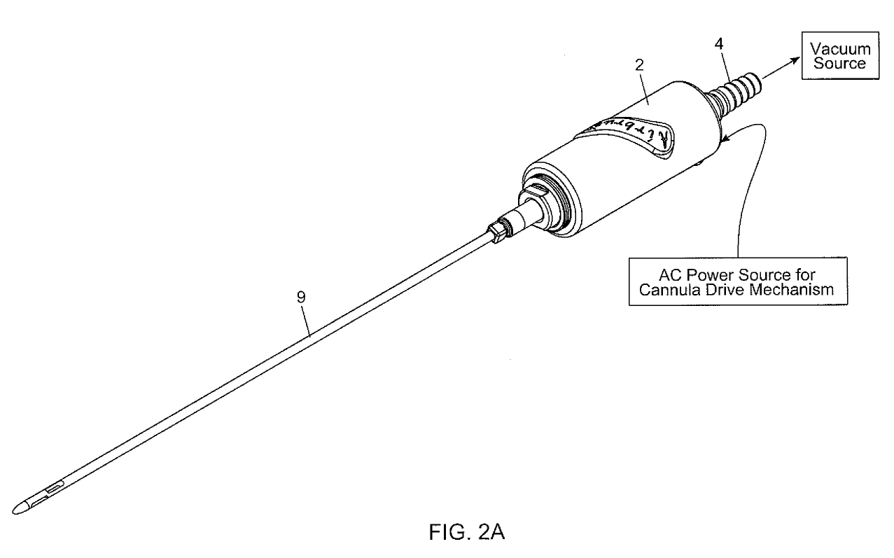

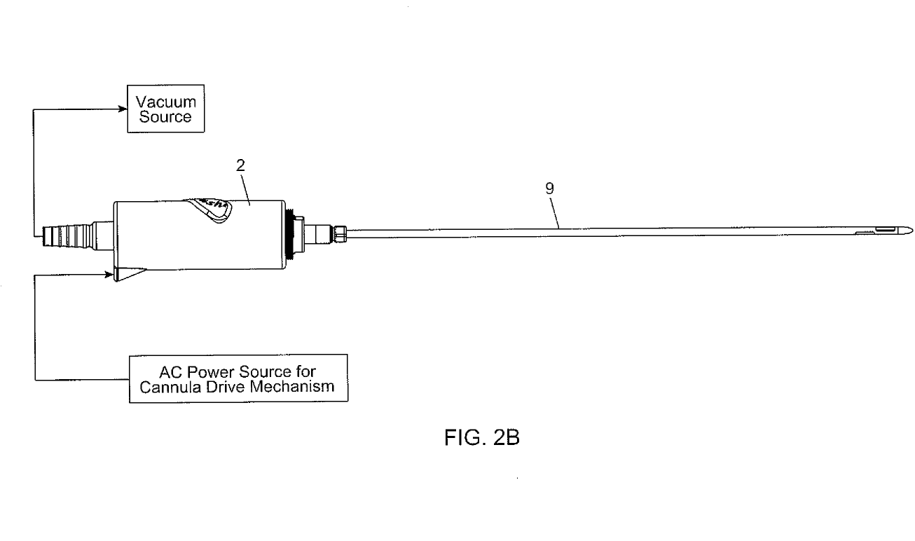

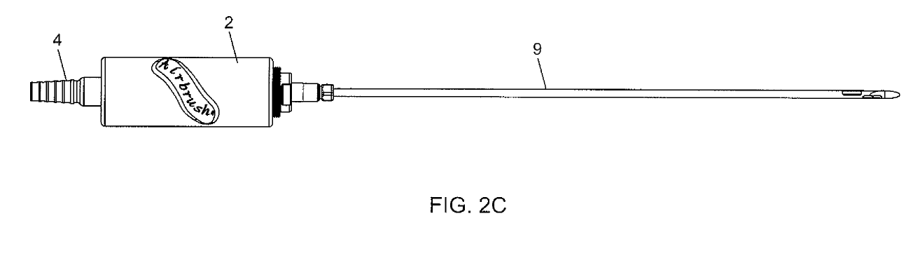

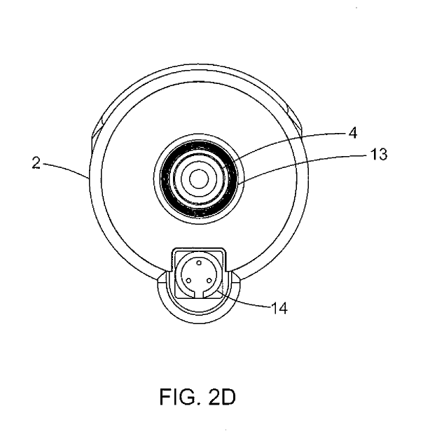

Referring now to FIGS. 2A through 5E, the first illustrative embodiment of the tissue aspiration instrumentation system of the present invention will be described.

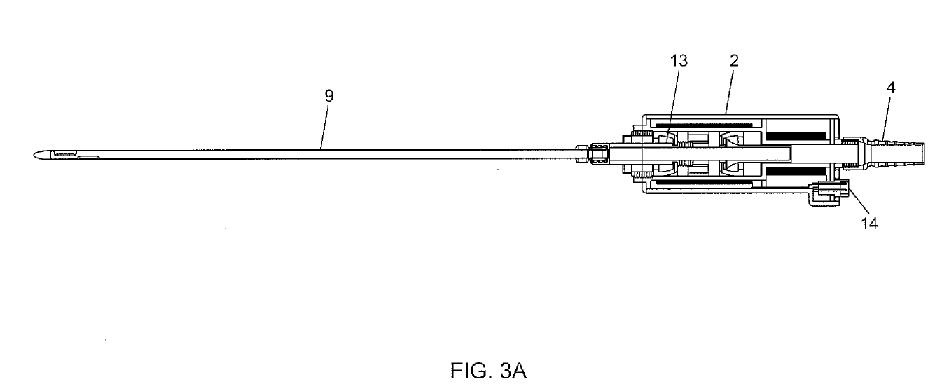

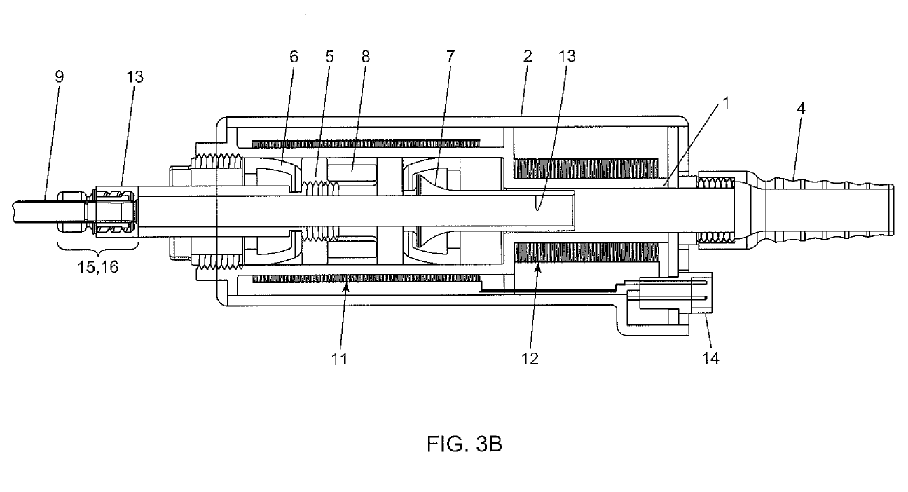

As shown in FIGS. 2A through 3A, the first illustrative embodiment of the tissue aspiration instrumentation system of the present invention comprising: a hand-supportable tissue aspiration instrument having (i) a hand-supportable housing with a stationary tubing connector provided at the rear of the housing and receiving a length of flexible tubing connected to a vacuum source. The single-cannula assembly has an inner cannula coupled to an electromagnetic-based cannula drive mechanism disposed within the hand-supportable housing and powered by an AC electrical signal power source, while its stationary outer cannula is releasably connected to the front portion of the hand-supportable housing.

As shown in FIG. 3B, the hand-supportable tissue aspiration instrument of FIGS. 2A and 3A, comprises a cylindrical guide tube 1 mounted within the hand-supportable housing 2, and the (disposable) cannula base portion 13 carries a permanent magnetic ring 8 between a set of fluid seals 6 and 7 that slidably support the cannula base portion 13 within the cylindrical guide tube. As shown, the cannula 9 is coupled to the cannula base portion 13 by way of a mated leur-lock coupling 15, 16, and the lumen extending within the cannula and its base portion is in fluid communication with the stationary tubing connector 3, by way of the interior volume of the cylindrical guide tube 1 between the cannula base portion 13 and the stationary tubing connector 4. The stationary tubing connector 3 (having a barbed tubing connector portion) is adapted to unscrew from the rear portion of the hand-supportable housing so that housing back plate 3 can be removed so that the cylindrical guide tube (i.e. the wound bobbin) can be slid into the hand-supportable housing 2. The top and bottom of the hollow cylindrical ring magnet 8 produce opposing magnetic poles, and magnet 8 is secured onto the cannula base portion 13 by way of nut 5 which screws onto a set of threads form on other surface of the cannula base portion. In the illustrative embodiment, the fluid seals 6, 7 are realized as a pair of thin-walled, collapsible (i.e. invertible) bell-shaped silicone sealing washers which act as front and rear diaphragms allowing motion of the cannula base portion within the cylindrical guide tube. By setting mid-point geometry, one washer can effect a return stroke without need of coil polarity reversal, simply pulsing sufficing. Mounted about outer surface of the cylindrical guide tube, front and rear coil windings 11 and 12 are formed, respectively, and electrically connected to the connector plug 14 formed on the rear end of the hand-supportable housing.

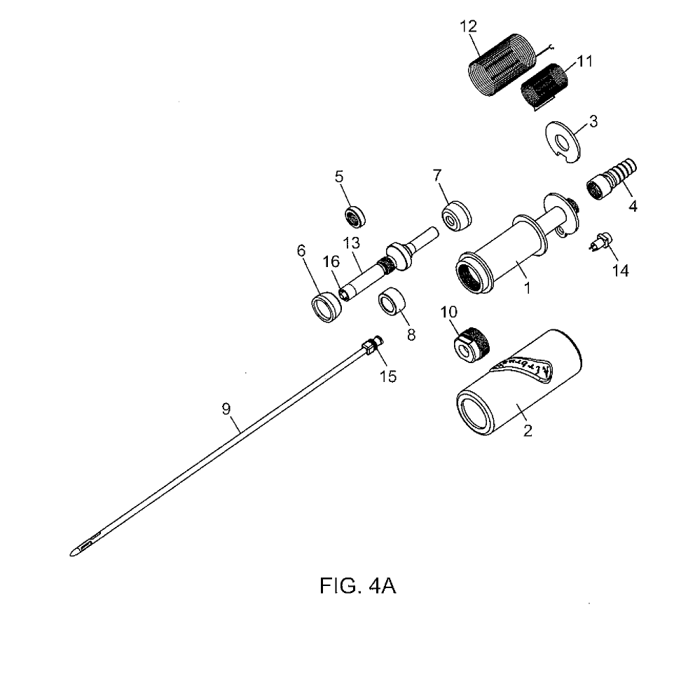

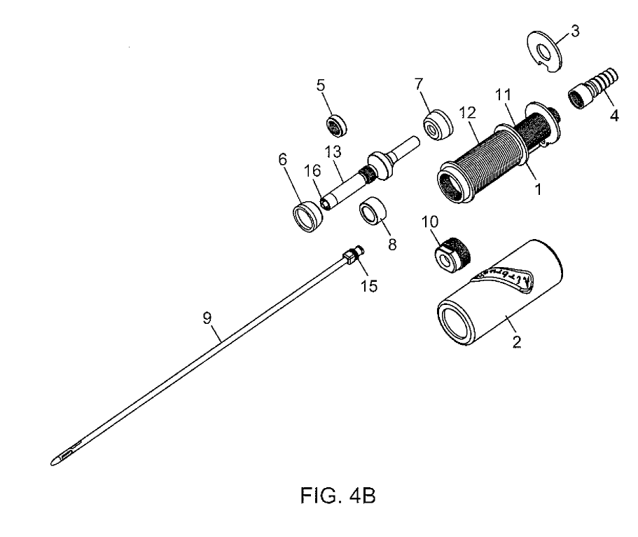

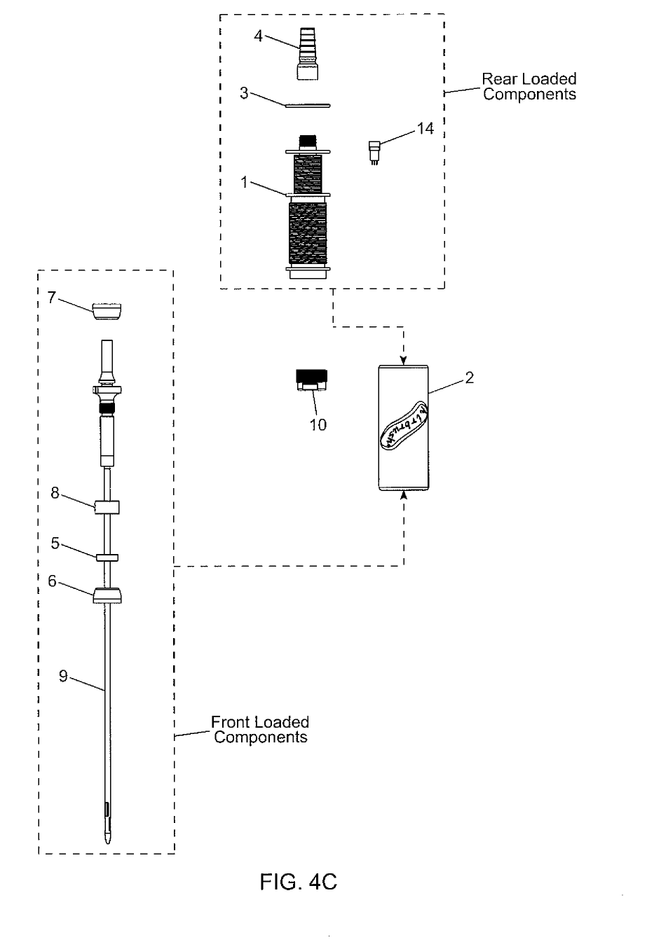

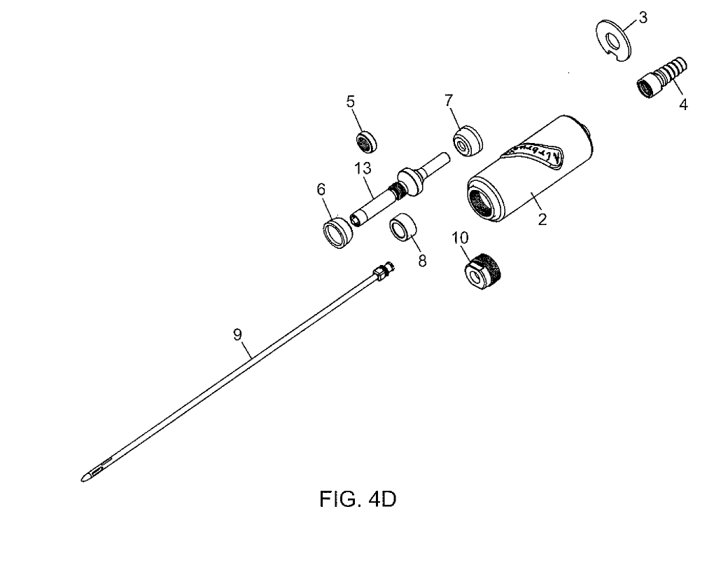

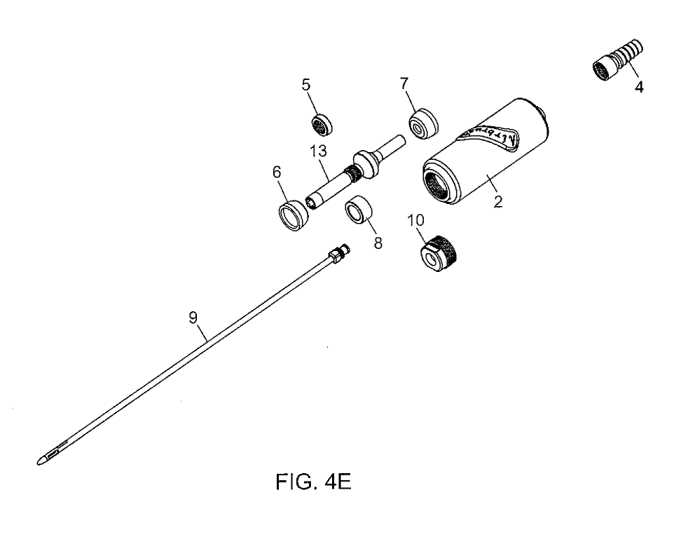

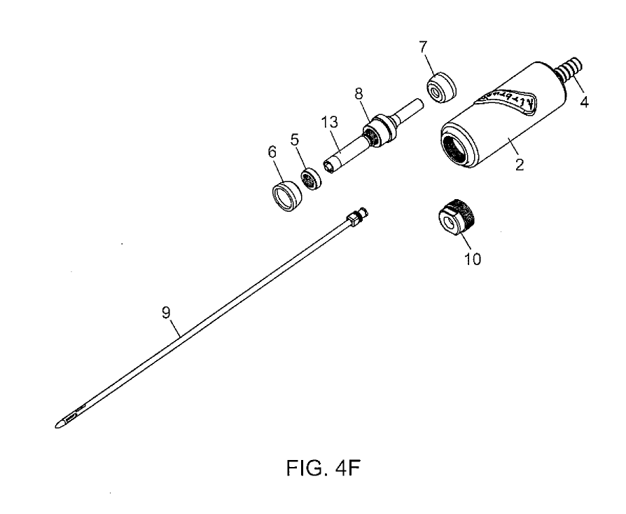

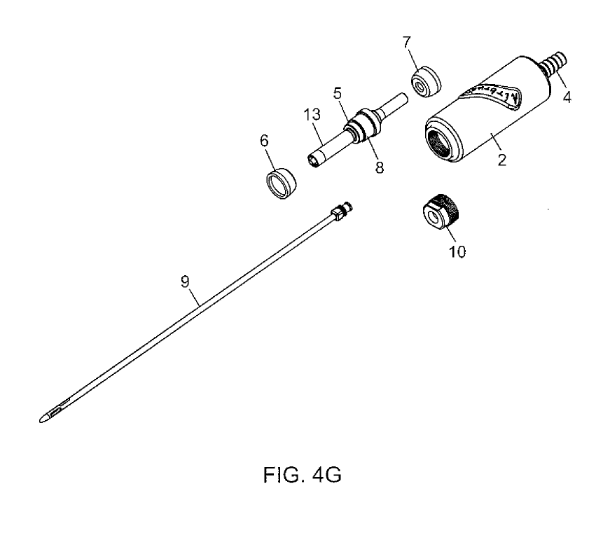

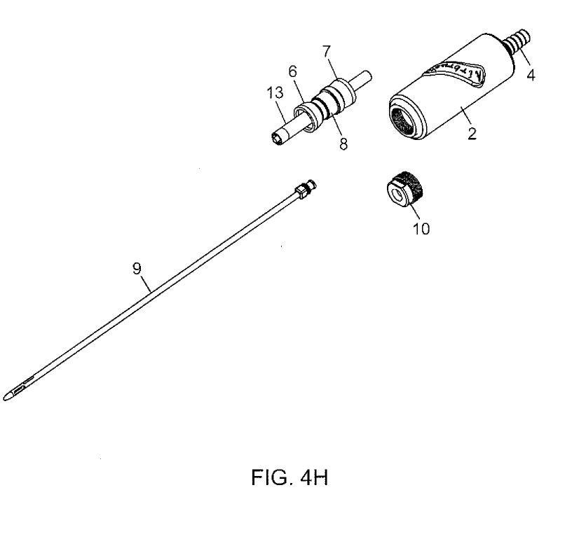

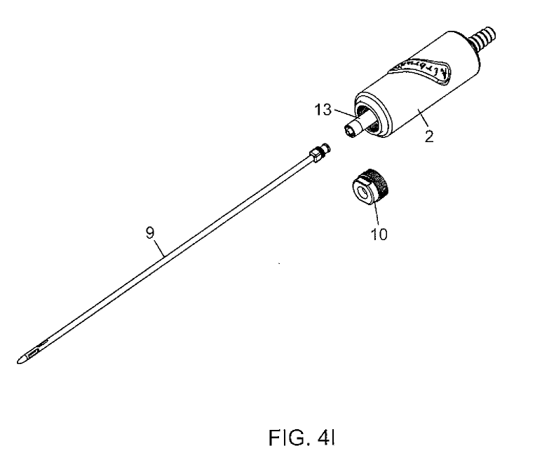

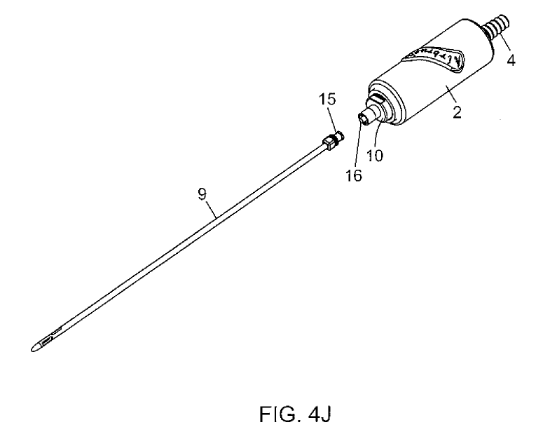

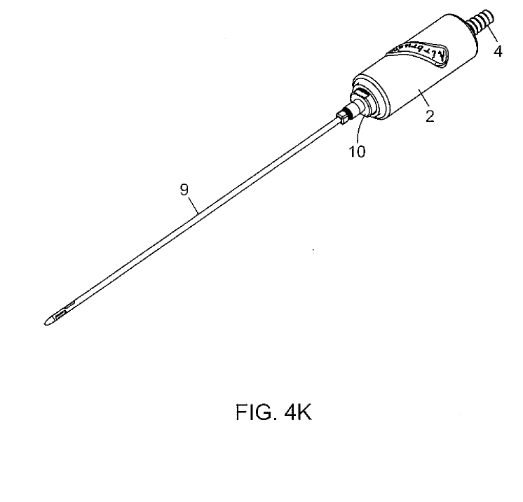

FIG. 4A shows a fully exploded view of the hand-supportable tissue aspiration instrument of FIGS. 2A and 2B, clearly revealing its dissembly of components, as comprising: cylindrical guide tube 1 with flanges for containing electromagnetic coil windings (11, 12), a hand-supportable housing 2, housing back plate 3, stationary tubing connector 4 with a vacuum tubing barb, a magnet fastening nut 5, a front washer 6, a back washer 7, a ring magnet 8, a cannula 9 provided with a luer lock fastener 15, a front chamber screw cap 10, a back electromagnetic coil 11, a front electromagnetic coil 12, a disposable cannula base portion 13 realized as luer lock fastener, a contact/connector plug 14 (e.g. Binder 719), a (male) luer lock fitting 15, and a (female) luer lock fitting 16. FIGS. 4B through 4I show how these components are assembled in step order fashion, as during manufacture on an assembly line. FIG. 4J shows that after the hand-held instrument is assembled, its cannula assembly 9 is simply connected to the installed (disposable) cannula base portion 13, using a luer lock coupling mechanism 15, 16 well known in the art, to completely assemble the instrument and prepare it for use in surgery.

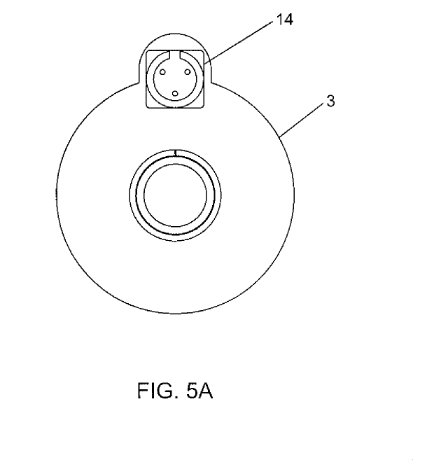

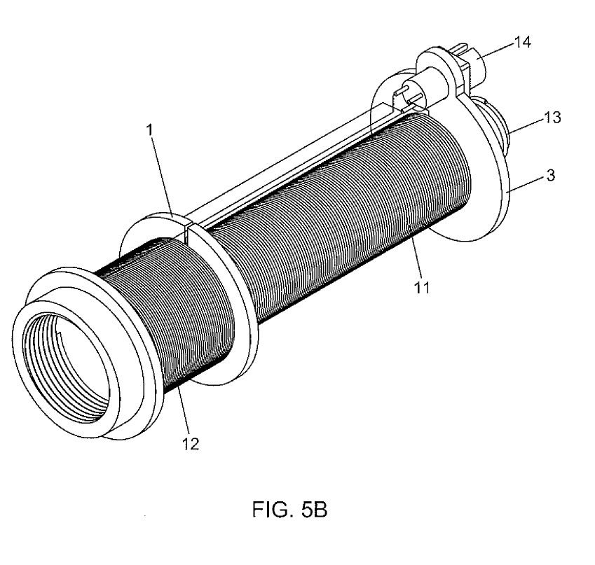

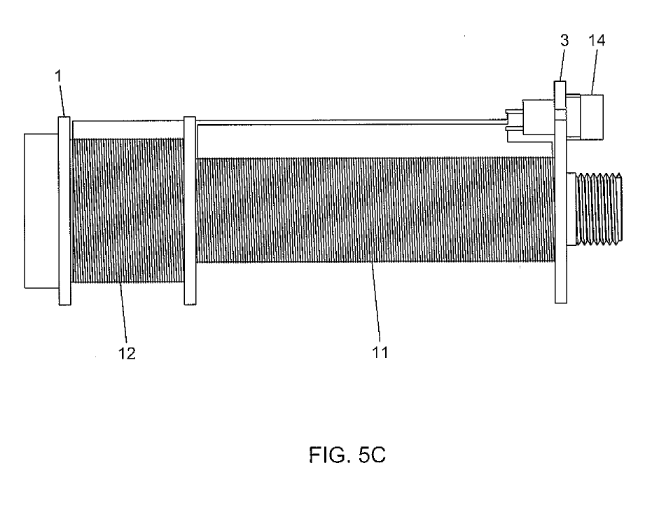

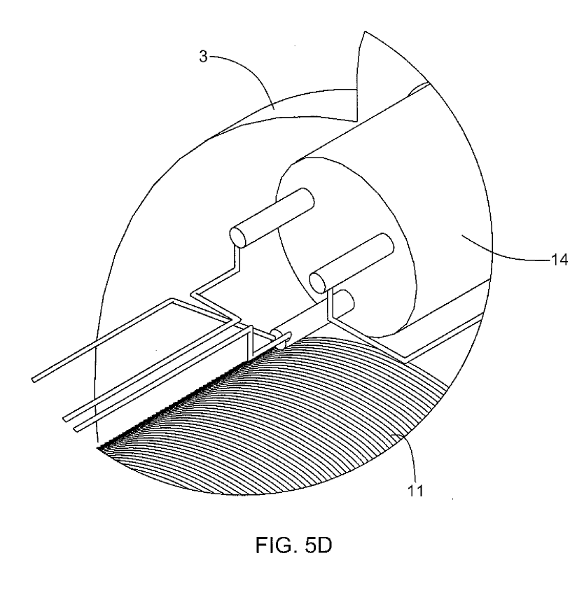

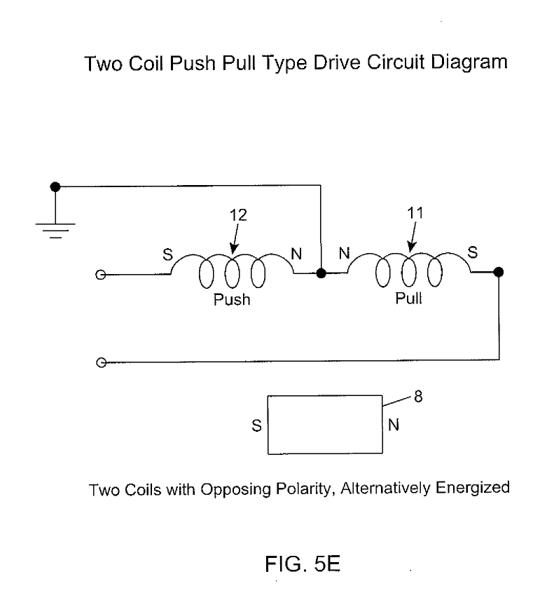

Taken together, FIGS. 5A, 5B 5C and 5D shows how the first and second electromagnetic coils 11, 12 are wound about the cylindrical guide tube 1, and then how wiring of these coils are electrically connected to the electrical connector mounted on the housing back plate 3, employed in the first illustrative embodiment shown in FIGS. 2A through 5E. FIG. 5E shows the schematic diagram depicting how the two coil 11 and 12 are driven by a push-pull type of circuit, for the purpose of enabling the cannula drive mechanism employed in the hand-supportable tissue aspiration instrument of the present invention illustrated in FIG. 3B.

### Second Illustrative Embodiment of the Tissue Aspiration Instrumentation System of the Present Invention

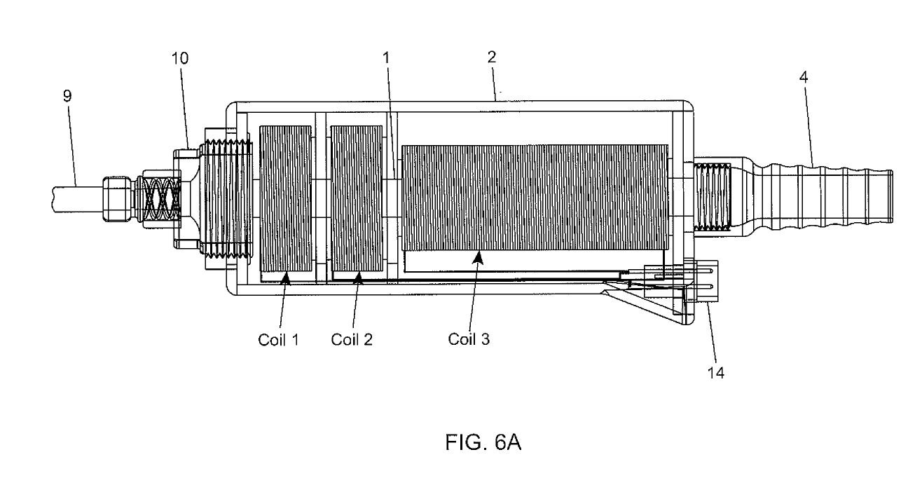

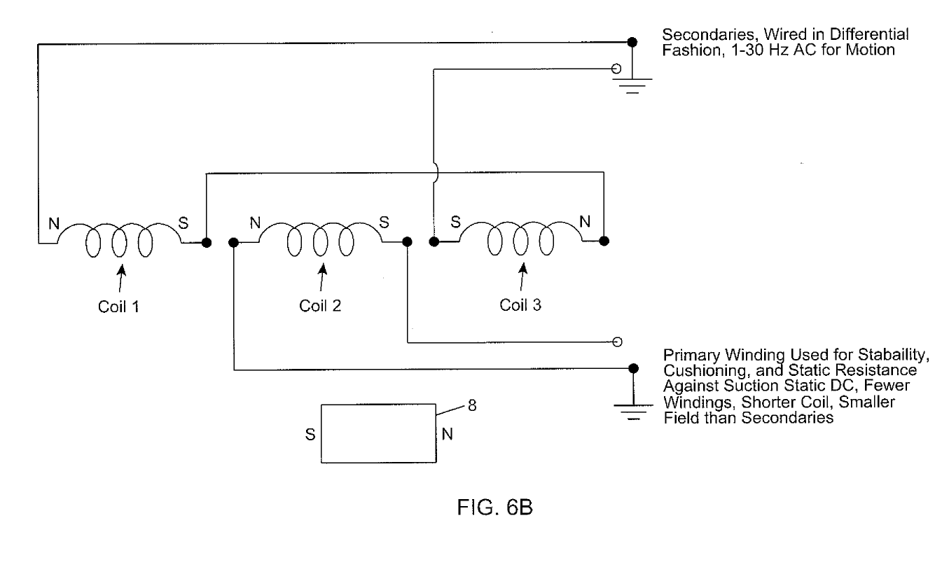

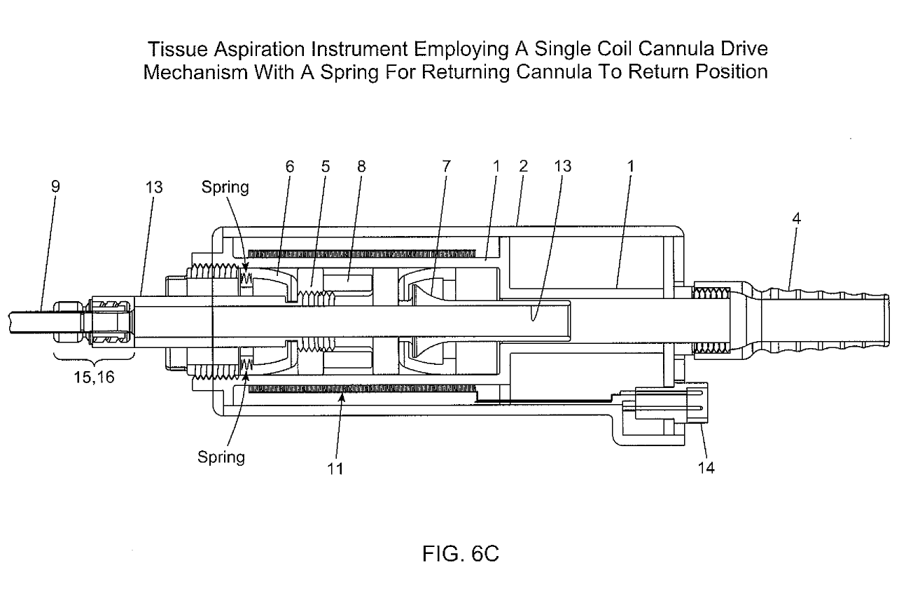

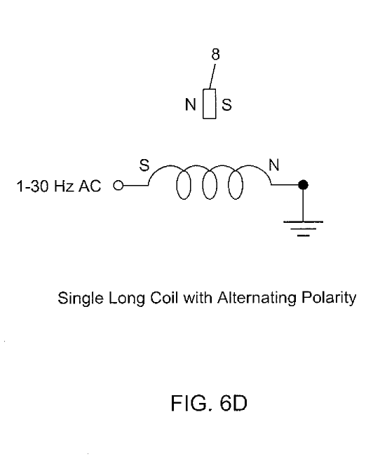

In FIG. 6A, a second embodiment of the hand-supportable tissue aspiration instrument of FIGS. 2A and 2B is show, comprising a cylindrical (cannula base portion) guide tube 1′ adapted to support three electromagnetic coils, rather than two coils used in the first illustrative embodiment, for the purpose of implementing the cannula drive mechanism employed in the tissue aspiration instrument. FIG. 6B shows the schematic diagram for this three coil push-pull type of circuit, driven by a 1-30HS AC electrical signal, for enabling the cannula drive mechanism employed in the alternative embodiment of the hand-supportable tissue aspiration instrument of the present invention illustrated in FIG. 6A. In all other respects, the tissue aspiration instrument of the second illustrative embodiment is like the tissue aspiration instrument of the first illustrative embodiment.

### Third Illustrative Embodiment of the Tissue Aspiration Instrumentation System of the Present Invention

In FIG. 6C, a third embodiment of the hand-supportable tissue aspiration instrument of the present invention is shown comprising a cylindrical (cannula base portion) guide tube 1″ supporting a single electromagnetic coil used to realize the cannula drive mechanism employed in the tissue aspiration instrument. FIG. 6D shows the schematic diagram of this single coil type circuit, driven by an alternating polarity electrical signal, for enabling the cannula drive mechanism employed in the alternative embodiment of the hand-supportable tissue aspiration instrument of the present invention illustrated in FIG. 6A. In all other respects, the tissue aspiration instrument of the third illustrative embodiment is like the tissue aspiration instrument of the first illustrative embodiment.

### Fourth Illustrative Embodiment of the Tissue Aspiration Instrumentation System of the Present Invention

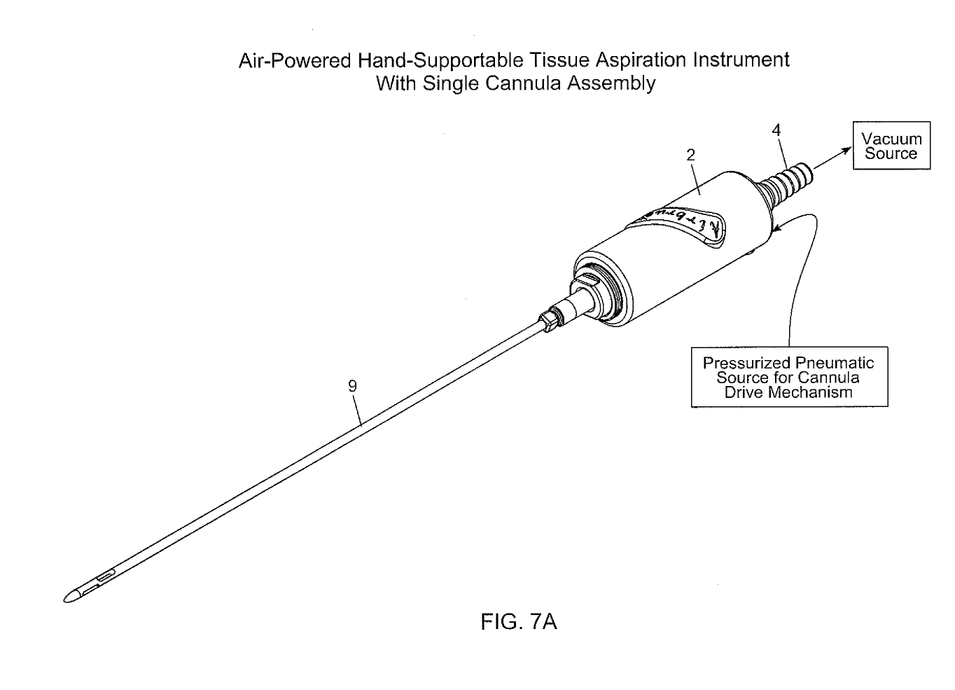

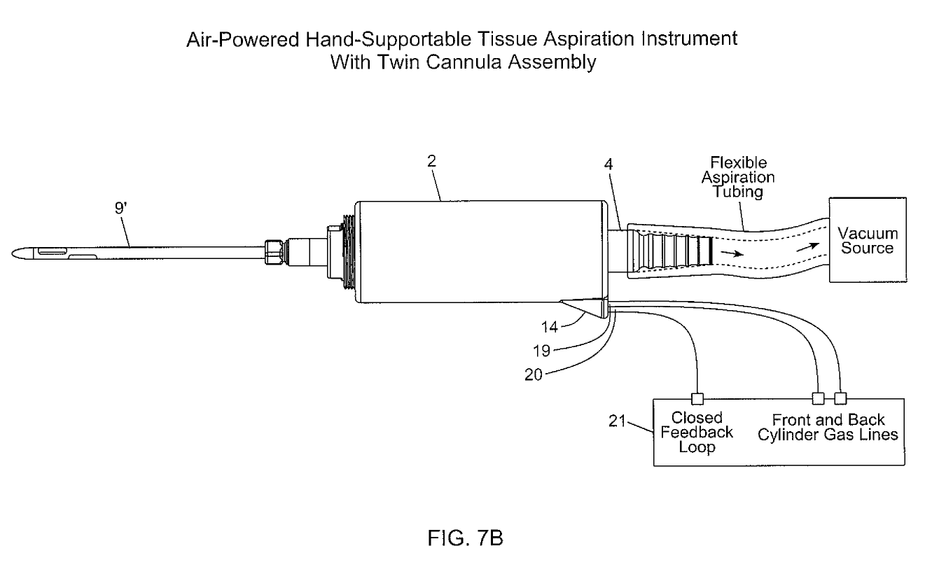

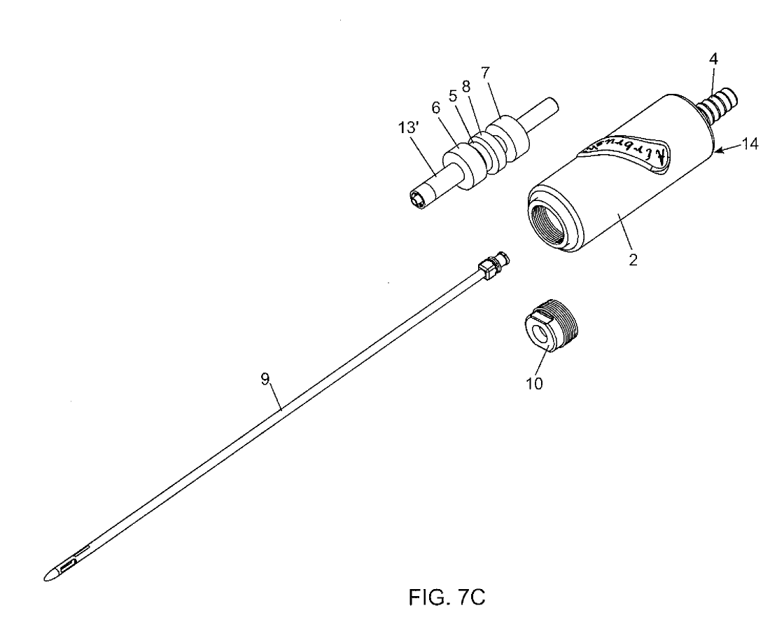

FIG. 7A shows a fourth illustrative embodiment of the tissue aspiration instrumentation system of the present invention, comprising: a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a single-cannula assembly having an inner cannula coupled to an pneumatically-powered cannula drive mechanism disposed within the hand-supportable housing and powered by a source of pressurized air or other gas, while its stationary outer cannula is releasably connected to the front portion of the hand-supportable housing;

As shown in FIG. 7B, the air-powered tissue aspiration instrument of FIG. 7A comprises a single-button quick connect plug, and associated multi-core cable assembly is provided on the rear portion of the hand-supportable housing, for supporting two gas lines and three electric wires between the instrument and its controller in a single bundle, as taught in U.S. Pat. No. 7,381,206 to Cucin, incorporated herein by reference, but without the extra two widely separated RF leads provided for electro-cautery and without the extra 3 pins for LV control circuits;

As shown in FIG. 7C, shows the fourth illustrative embodiment of the tissue aspiration instrumentation system of the present invention as comprising a hand-supporting housing, in which its cylindrical (cannula base portion) guide tube and air-powered driven mechanism are installed, while its cannula base portion, cannula and cannula lock nut are shown disassembled outside of the hand-supportable housing;

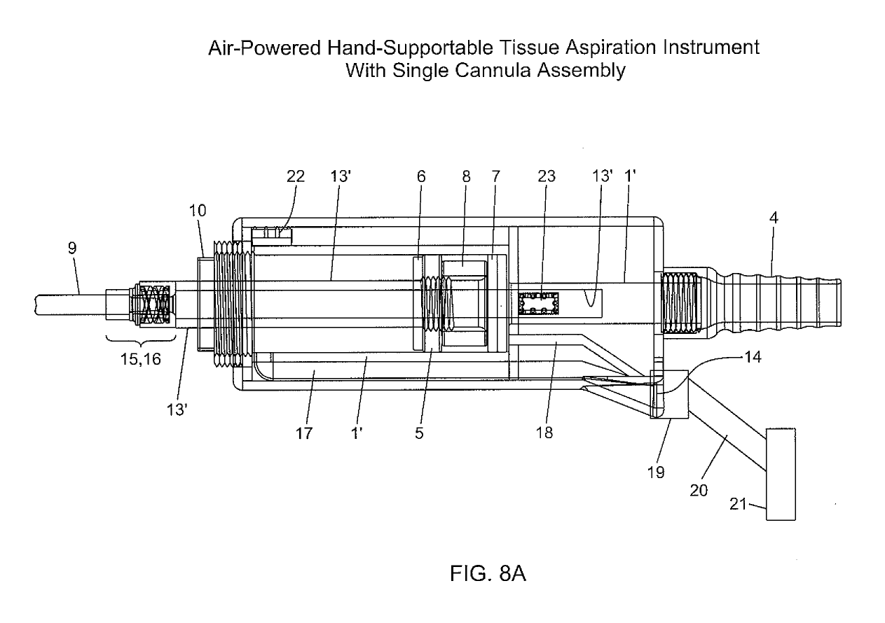

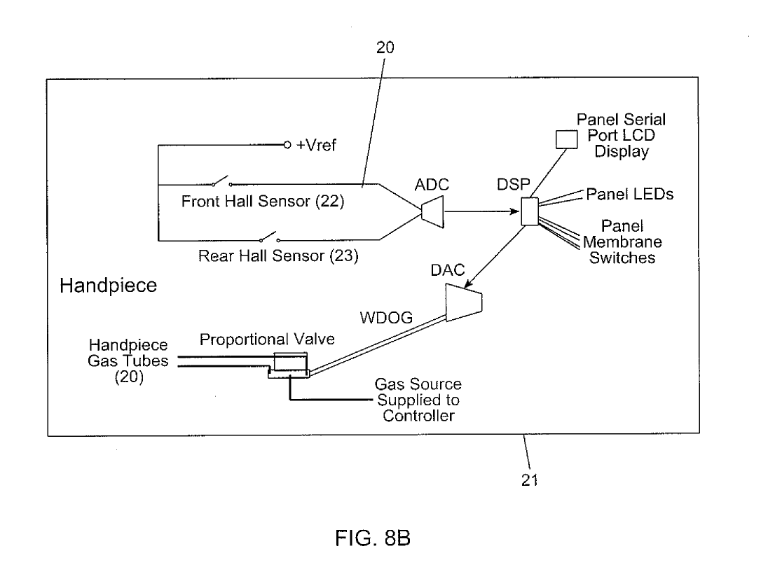

In FIG. 8A, the hand-supportable tissue aspiration instrumentation system of FIG. 8A is shown configured with its aspiration source, its controller and pneumatic power source, and multi-core cable assembly.

FIG. 8A also reveals a number of important features of this illustrative embodiment of the tissue aspiration instrument, namely: solitary reciprocating (inner) cannula 9 has luer lock fitting 15 to mate to luer lock fitting 16 on cannula drive mechanism 13; magnet 8 is affixed to cannula base portion using a screw-on nut 5; front and rear gas tubes 17 and 18 run to from the front of the housing to the rear multi-core quick connect plug 19; the quick connect multi-core plug 19 connects to multi-core cable containing two fluidic (gas) channels 20 and at least 3 low voltage electrical circuits; the cable 20 runs to controller 21 within which the gas channels directly attached to the compressed gas source (not shown); the front and rear Hall sensors 22 and 23 are provided within the hand-supportable housing, for detecting the excursion of the cannula base portion 13 within the cylindrical guide tube 1; front and rear flat sealing washers 6 and 7 are provided for slidably supporting the cannula base portion 13 along the cylindrical guide tube 1; threaded chamber cover 10 is provided with a hole, through which the cannula 9 protrudes; sufficiently large through-and-through vents are formed in the threaded chamber cover 10 to allow any gas that leaks past the front washer 6, to exit the chamber in single cannula embodiment (in the twin cannula embodiment. Such air venting to the ambient is less critical because the concentric tube-with-a-tube structure, and the sliding of the cannula base portion 13 inside the rear tubing connector assembly, provides effective seals in and of themselves.

In FIG. 8B, the controller (and air-power supply) console 21 shown in the hybrid schematic diagram of FIG. 8A, is shown comprising a number of components, namely: an ADC receiving signals generated by the front and rear Hall-effect cannula base position sensors installed within the hand-supportable housing of the instrument; a LCD panel; communication ports; LED indicators; and panel membrane switches supported on the controller console housing; digital signal processor (DSP); and a DAC and proportional valve contained within the controller console housing, and supplying gas tubes (via the multi-code cable assembly); and ports for receiving a supply of pressurized gas, for controlled supply to the cannula drive mechanism of this embodiment of the present invention. The details of this controller 21 can be found in U.S. Pat. No. 7,381,206 to Cucin, incorporated herein by reference.

### Fifth Illustrative Embodiment of the Tissue Aspiration Instrumentation System of the Present Invention

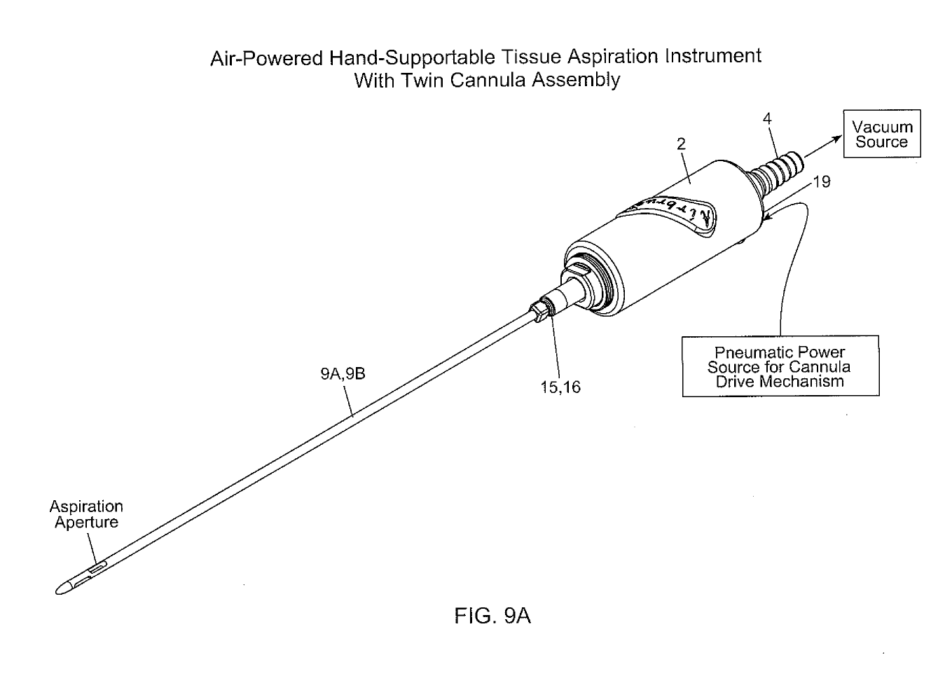

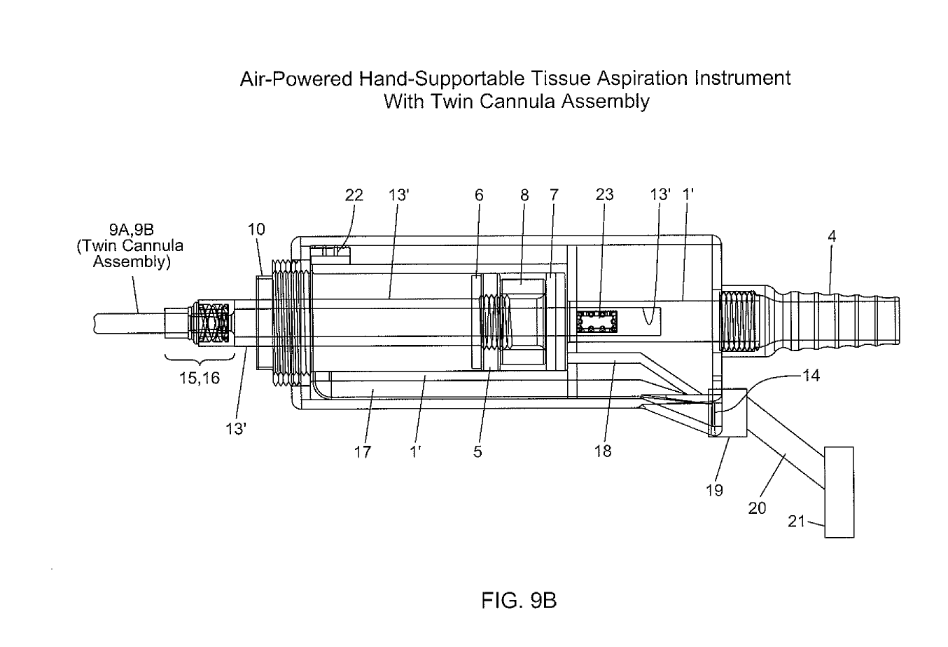

FIG. 9A shows a fifth illustrative embodiment of the tissue aspiration instrumentation system of the present invention, comprising: a hand-supportable tissue aspiration instrument having (i) a hand-supportable housing with a stationary tubing connector provided at the rear of the housing and receiving a length of flexible tubing connected to a vacuum source and connecting to the cylindrical cannula base portion guide tube, and (ii) a twin-cannula assembly having an inner cannula coupled to an pneumatically-powered cannula drive mechanism disposed within the hand-supportable housing and powered by a source of pressurized air or other gas, while its stationary outer cannula is releasably connected to the front portion of the hand-supportable housing;

As shown in FIG. 9B, the air-powered tissue aspiration instrument of FIG. 9A, further comprises: a single-button quick connect plug 19, and associated multi-core cable assembly 20 is provided on the rear portion of the hand-supportable housing. The function of the multi-core cable assembly is to support at least two gas lines and at least three electric wires between the instrument and its controller 21 in a single bundle, as taught in U.S. Pat. No. 7,381,206 to Cucin, incorporated herein by reference, but without the extra two widely separated RF leads provided for electro-cautery and without the extra 3 pins for low voltage control circuits. Also, in this embodiment, the walls of at least the front (pneumatic) chamber portion of housing should be made from a non-magnetizable metal (e.g. SS 304) or other material that will support the necessary gas pressure of actuation (e.g. ˜100 PSI).

Also, the Hall effect sensors installed in the housing sense the position of the cannula base portion by sensing the magnetic field of its magnetic ring 8. As the cannula base portion 13′ reciprocates within the cylindrical guide tube 1′, the aspiration/vacuum tubing connected to the barb connector on the stationary tubing connector, remains stationary and thereby preventing any jerking action on the surgeon's hands which can cause carpal tunnel syndrome. Also, the inner and outer cannulas 9A, 9B are provided with luer-lock fittings 15, 16, while the cannula base portion is provided as a sterile single-use disposable item, made from plastic or metal, and having a low cost magnet and silicone washers to provide fluid seals between the cannula base portion and the cylindrical guide tube within the hand-supportable housing.

In this illustrative embodiment, there must be an air-tight seal around the (inner) cannula as it exits the pneumatic cylinder/chamber so that air pressure is not lost to the ambient environment. Any air will escape that seal and harmlessly vent into the air as the pneumatic cylinder is separate from the aspiration path (lumen) within the inner cannula. There must be a generous vent formed in the outer cannula base portion to make sure that any escaping air from the pneumatic chamber seal does not cross the space between the outer and inner cannulas into the patient during instrument operation. A second sealing washer distal to that vent may be employed for extra patient safety.

### Sixth Illustrative Embodiment of the Tissue Aspiration Instrumentation System of the Present Invention

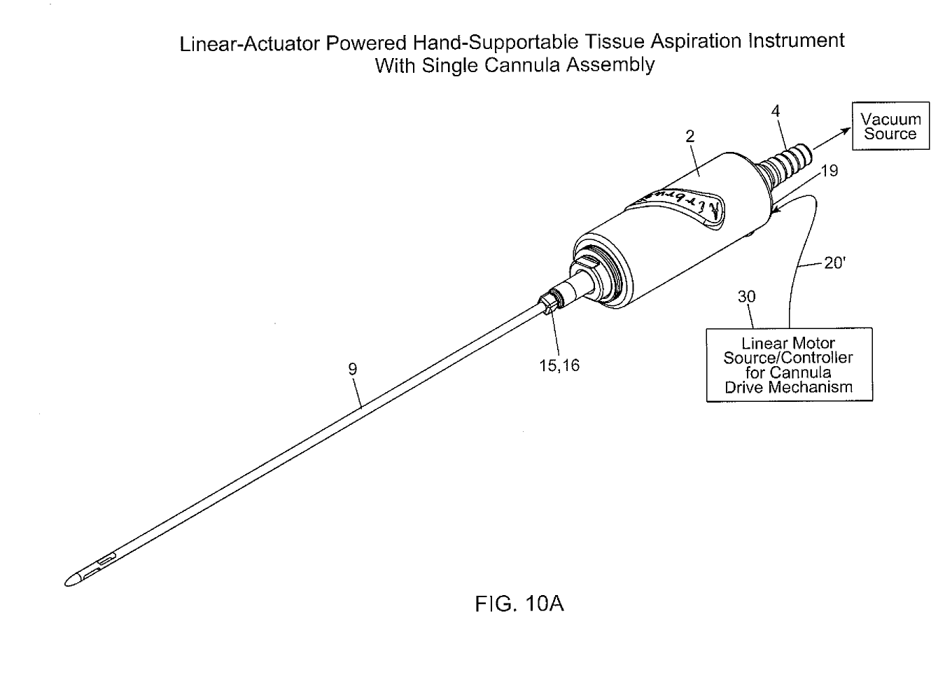

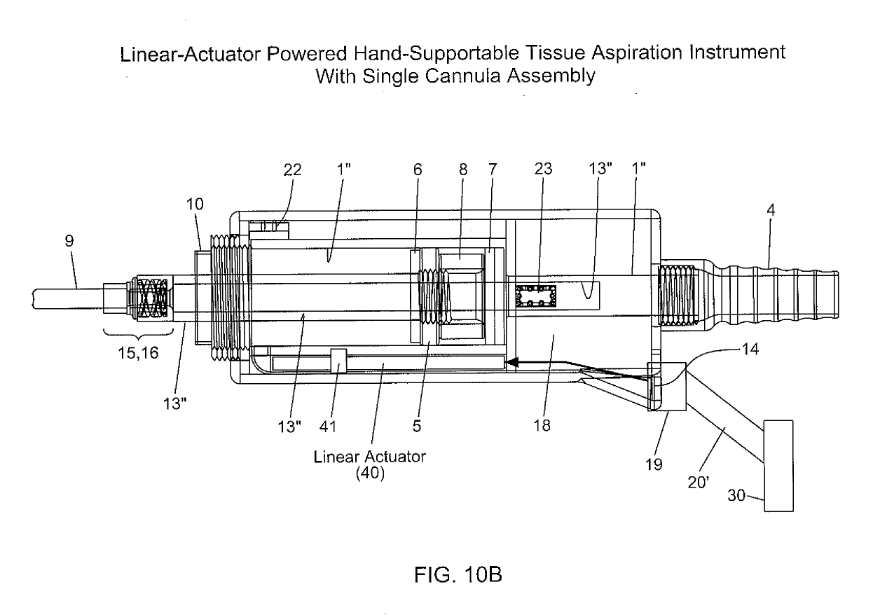

FIG. 10A shows a sixth illustrative embodiment of the tissue aspiration instrumentation system of the present invention, comprising a hand-supportable tissue aspiration instrument and a single-cannula assembly. The hand-supportable tissue aspiration instrument has a hand-supportable housing with a stationary tubing connector provided at the rear of the housing and receiving a length of flexible tubing connected to a vacuum source and connecting to the cylindrical cannula base portion guide tube. The single-cannula assembly has an inner cannula coupled to an linear-actuator powered cannula drive mechanism disposed within the hand-supportable housing and powered by a source of electrical power, while its stationary outer cannula is releasably connected to the front portion of the hand-supportable housing;

As shown in FIG. 10B, the cannula drive mechanism provided within the air-powered tissue aspiration instrument of FIG. 10A comprises a linear actuator (i.e. linear motor) 40 mounted within the hand-supportable housing, outside of the cylindrical (cannula base portion) guide tube 1″ adapted with a permanent magnet 41. During operation, the linear actuator is driven by electrical power signals supplied through a quick-release type connector, connecting a flexible electrical signal cable between the instrument and its controller console 30, and the magnet 41 along the linear actuator 40 moves along the length of the cylindrical guide tube, and coaxially exerts forces on the magnetic ring 8 on the cannula base portion 13″ of the cannula assembly. In nearly all other respects, this sixth illustrative embodiment of the instrument of the present invention is similar to the fifth illustrative embodiment shown in FIG. 9B.

### Seventh Illustrative Embodiment of the Tissue Aspiration Instrumentation System of the Present Invention

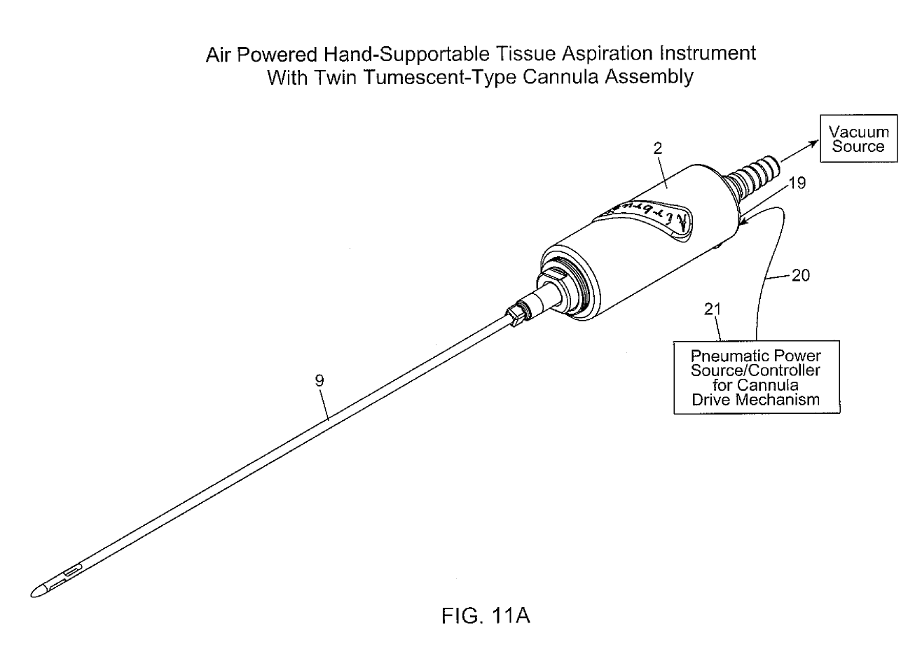

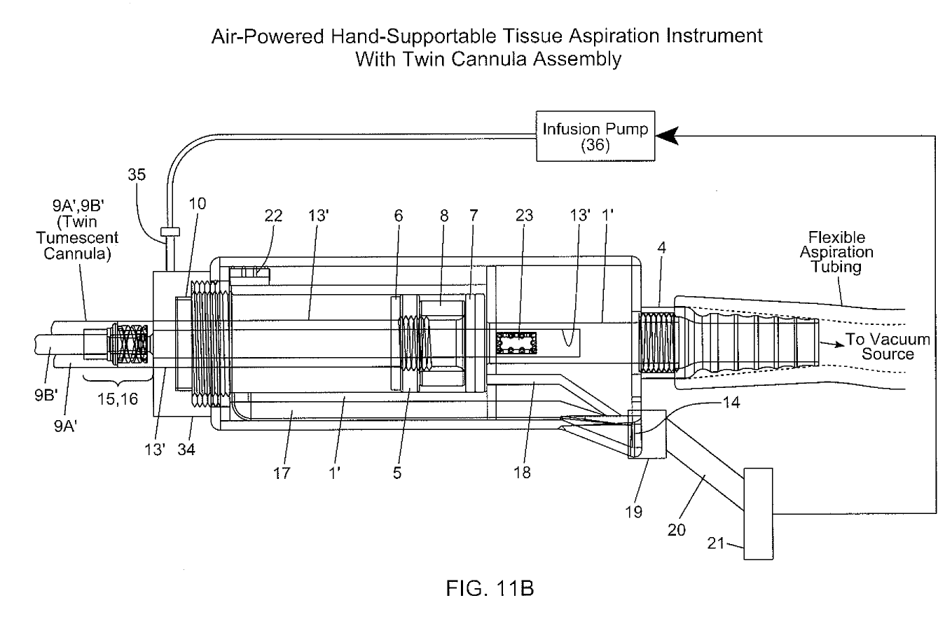

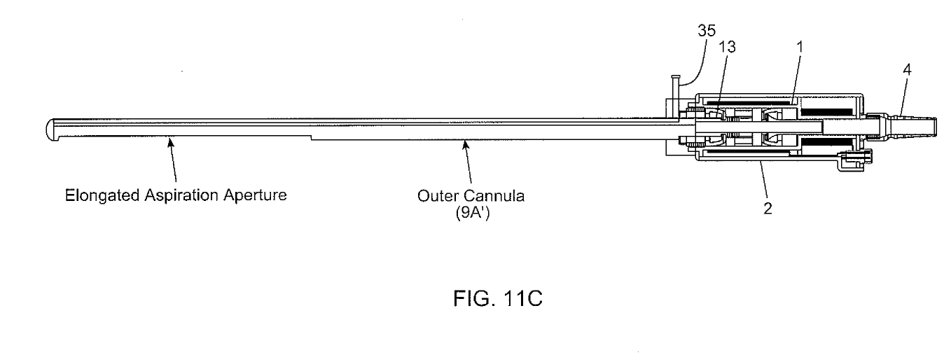

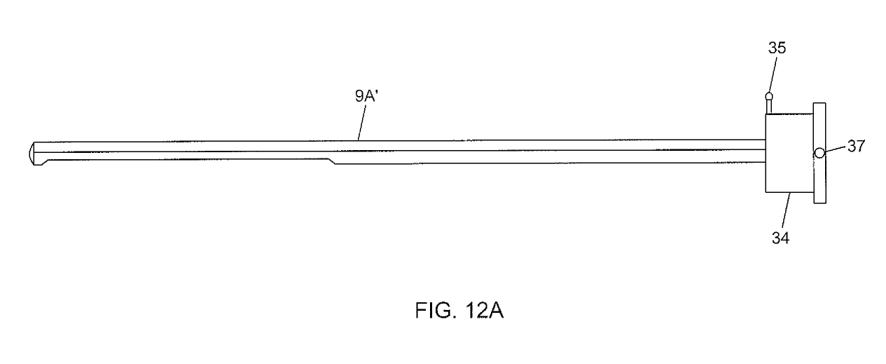

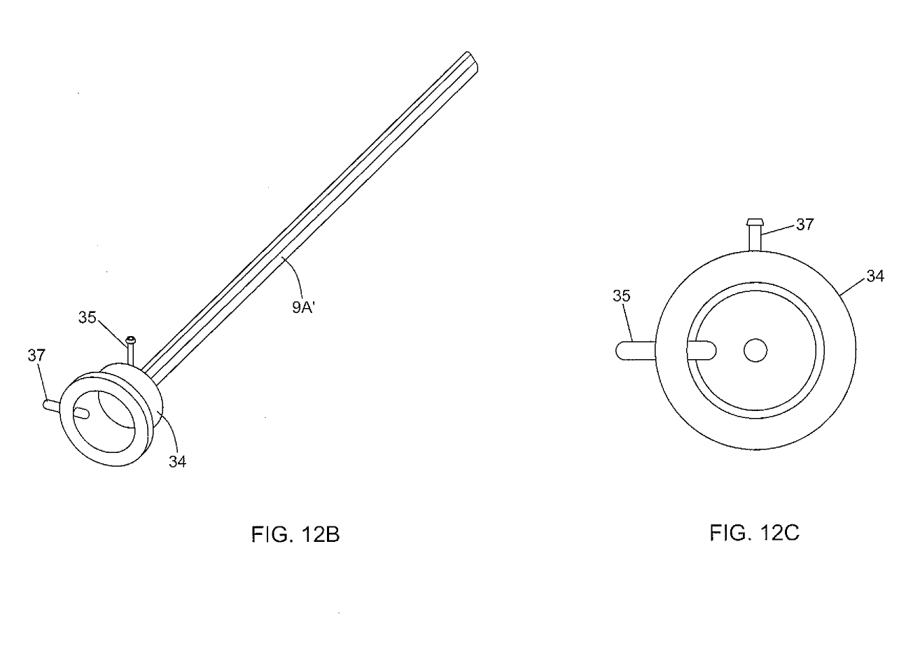

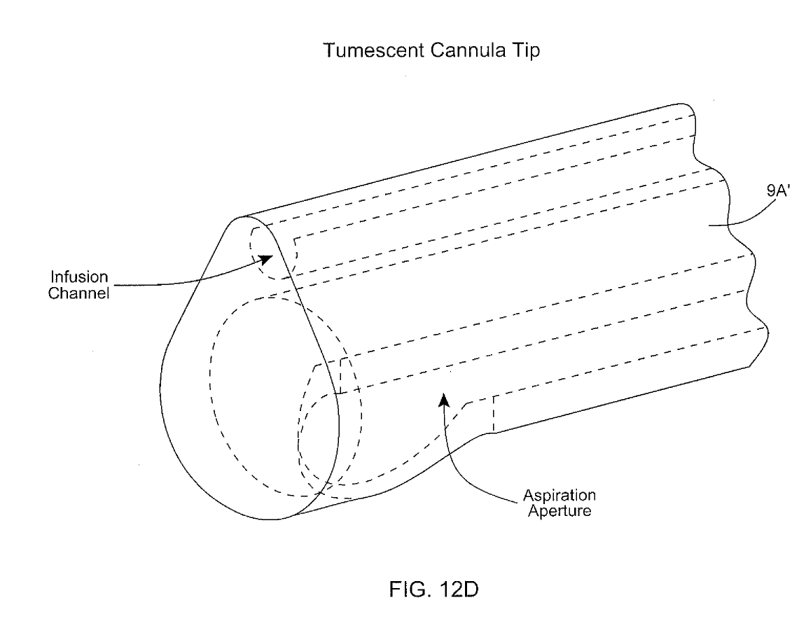

FIG. 11A shows a seventh illustrative embodiment of the tissue aspiration instrumentation system of the present invention, as comprising: a hand-supportable tissue aspiration instrument and a twin tumescent-type cannula assembly 9″, as shown in FIGS. 12A through 12D. The hand-supportable tissue aspiration instrument has hand-supportable housing with a stationary tubing connector 4 provided at the rear of the housing 2 and receiving a length of flexible tubing connected to a vacuum source and connecting to the cylindrical cannula base portion guide tube 1′. The twin tumescent-type cannula assembly has an inner cannula coupled to an pneumatically-powered cannula drive mechanism disposed within the hand-supportable housing and powered by a source of pressurized air or other gas, while its stationary outer cannula is releasably connected to the front portion of the hand-supportable housing. In nearly all other respects, except for the twin tumescent cannula assembly, this seventh illustrative embodiment of the instrument of the present invention is similar to the fifth illustrative embodiment shown in FIG. 9B.

As shown in FIG. 11B, the air-powered tissue aspiration instrument of FIG. 11A further comprises an aspiration source, a controller 21, a pneumatic power source, and a multi-core cable assembly 20, and a tumescent outer cannula infusion port 35 connected to an infusion pump 36, that is daisy chained to the controller 21 for synchronized pulse control, and operational to synchronize the release of irrigation fluid (i.e. infusion) with inner cannula motion.

FIG. 11C shows air-powered tissue aspiration instrument of FIGS. 11A and 11B, wherein the tumescent outer cannula 9A′ is attached to the chamber screw cap 10, allowing the base portion 34 of the outer cannula 9B′ to be screwed on same threads of the chamber screw cap 10. Preferably, the chamber screw cap will be a larger size luer-lock style fitting so as to assure registration of the elongated aspiration aperture (i.e. slot) formed at the distal end of the outer cannula, and the aspiration aperture(s) formed in the distal portion of the inner cannula. Such registration ensures the reciprocally-driven inner cannula is “keyed” and cannot rotate within the outer cannula, and remains in constant alignment during operation.

FIG. 12B shows a tab 37, provided on the base portion of the outer cannula, for fastening the outer cannula over the inner cannula, and aligning the outer cannula with respect to the inner cannula.

FIG. 12C shows the base portion of the outer cannula shown in FIG. 12B, and FIG. 12D shows, in greater detail, the tumescent cannula tip portion of the tumescent outer cannula employed in the instrument shown in FIGS. 11A and 11B.

### Alternative Embodiments which Readily Come to Mind

While the tumescent cannula shown in FIGS. 12A through 12D has been shown used with a twin cannula assembly, it is understood that in alternate embodiments, the inner cannula can be adapted to provide a similar fluid infusion channel that terminates proximal to the luer fitting and allows for fluid infusion. As indicated, in twin cannula embodiments, infusion can be either synchronized. However, in single cannula embodiments, infusion can be unsynchronized as there will be less advantage and practicality in providing synchronization in a more rapidly reciprocating, short stroke single cannula instrument design.

While a barb Christmas-tree type connector is shown on the stationary tubing connector, of each hand-supportable housing, it is understood that the stationary tubing connector may also be realized as a snap-lock type connector for establishing and maintaining a connection with the end portion of flexible aspiration tubing.

The single coil cannula drive mechanism can also be realized by pulsing a single electromagnetic coil and using a spring to return the cannula base portion to a return position, where it was location before the application of the magnetic field.

Alternatively, the single coil cannula drive mechanism can be realized using a Hall effect switch, or a physical switch, configured in series with the electric circuit which powers the coil to cause reciprocation of the cannula base portion.

Further, the powered tissue aspiration instrument of the present invention can be designed so that its cylindrical guide tube is made very simple, inexpensively and is disposable so as to eliminate the need for a magnet which can loose its strength with autoclaving. The cannula base portion can be made so as to use washers that are wafer thin, for only one day of surgery. Such washers can function as diaphragms, staying in place and deforming to allow to-fro motion of the cylindrical guide tube within the cylindrical guide tube. Also, these washers can have an umbrella-shape, or be have a thin cylindrical geometry.

Also, while not shown, any embodiment of the power-assisted liposuction instrument of the present invention can be provided with various means along the cannula assembly to effect hemostasis during liposuction procedures and the like using, for example, RF-based electro cauterization, as taught in Applicant's prior U.S. Pat. Nos. 6,872,199 and 7,381,206, incorporated herein by reference.

While the particular embodiments shown and described above have proven to be useful in many applications in the liposuction art, further modifications of the present invention disclosed herein will occur to persons skilled in the art to which the present invention pertains. All such modifications are deemed to be within the scope and spirit of the present invention defined by the appended claims.

1. A tissue aspiration instrument system comprising:
a hand-supportable tissue aspiration instrument including (a) a hand-supportable housing having (i) a front portion and a rear portion aligned along a longitudinal axis, (ii) a tubing connector mounted to the rear portion of said hand-supportable housing for connecting a first end of a section of flexible tubing to said tubing connector, so that a second end of said section of flexible tubing can be connected to a vacuum source, and (iii) a cannula drive mechanism; and
(b) a twin cannula assembly having
a hollow outer cannula with an elongated outer aspiration aperture and having a outer cannula base portion stationarily connected to the front portion of said hand-supportable housing, and
a hollow inner cannula with an inner aspiration aperture and disposed within said hollow outer cannula and having an hollow inner cannula base portion mounted within said hand-supportable housing for reciprocating movement, and
an irrigation channel is formed along said outer cannula, and in fluid communication with an irrigation aperture formed at the distal portion of said outer cannula and proximate to said elongated outer aperture; and
an infusion pump, operably connected to said outer cannula, for delivering controlled amounts of fluid to said irrigation channel; and
a controller for controlling said infusion pump and said cannula drive mechanism so that said fluid flows out of said irrigation aperture and proximate to said elongated aperture during the aspiration of visceral fat through said reciprocating inner aspiration aperture;
wherein said cannula drive mechanism causes (i) said hollow inner cannula base portion to reciprocate within said hand-supportable housing, (ii) said hollow inner cannula to reciprocate within said hollow outer cannula, and (iii) said inner aspiration aperture to reciprocate along said elongated outer aspiration aperture, while tissue is being aspirated through said elongated outer aspiration aperture and through said reciprocating inner aspiration aperture, and along a fluid communication channel extending from said inner aspiration aperture, along said hollow inner cannula and said hollow inner cannula base portion through said hand-supportable housing and into said tubing connector, and along said flexible tubing connected to said vacuum source.

2. The tissue aspiration instrument system of claim 1, wherein said hand-supportable housing further comprises:
an interior volume and a guide tube mounted within the interior volume, wherein said cannula drive mechanism is disposed adjacent said guide tube; and
a stationary tubing connector coaxially mounted to the rear portion of said hand-supportable housing along the longitudinal axis, connected to said guide tube, and having an exterior connector portion permitting said section of flexible aspiration tubing to be connected at its first end to said exterior connector portion, and where the second end of the section of flexible tubing is connected to said vacuum source.

3. The tissue aspiration instrument of claim 1, wherein said hollow inner cannula base portion comprises a tubular structure having a permanent magnet ring mounted about its outer surface and concentric with the longitudinal axis of said hollow inner cannula;
and wherein said cannula drive mechanism comprises at least one electromagnetic wire coil wound about said cylindrical guide tube, and for generating an electromagnetic force field that is driven by an electrical signal source, and electrically connected to an electrical signal source, for generating an electromagnetic force field which periodically pushes and pulls said permanent magnet ring and thereby causes (i) said hollow inner cannula base portion to reciprocate within said cylindrical guide tube, (ii) said hollow inner cannula to reciprocate within said hollow outer cannula, and (iii) said inner aspiration aperture to reciprocate along said elongated outer aspiration aperture.

4. The tissue aspiration instrument of claim 2, wherein said permanent magnet ring and said at least one electromagnetic coil form a magnetic coupling mechanism between said hollow inner cannula base portion and said guide tube.

5. The tissue aspiration instrument of claim 1, wherein said hollow inner cannula base portion comprises a tubular structure having a permanent magnet ring mounted about its outer surface and concentric with the longitudinal axis of said hollow inner cannula; and wherein said cannula drive mechanism comprises a pair of spaced apart electromagnetic wire coils wound about said cylindrical guide tube, and electrically connected to an electrical signal source, for generating an electromagnetic force field which periodically pushes and pulls said permanent magnet ring and thereby causes (i) said hollow inner cannula base portion to reciprocate within said guide tube, (ii) said hollow inner cannula to reciprocate within said hollow outer cannula, and (iii) said inner aspiration aperture to reciprocate along said elongated outer aspiration aperture.

6. The tissue aspiration instrument of claim 5, wherein said permanent magnet ring and said pair of spaced apart electromagnetic coils form a magnetic coupling mechanism between said hollow inner cannula base portion and said guide tube.

7. The tissue aspiration instrument of claim 2, wherein said stationary tubing connector comprises a barb-type connector to receiving and gripping said end portion of flexible aspiration tubing.

8. The tissue aspiration instrument of claim 2, wherein said stationary tubing connector comprises snap-lock type connector for establishing and maintaining a connection with said end portion of flexible aspiration tubing.

9. The tissue aspiration instrument of claim 1, wherein said guide tube is cylindrical shaped.

10. The tissue aspiration instrument of claim 1, wherein said infusion pump, operably connected to said outer cannula, delivers controlled amounts of fluid to said irrigation channel, over short periods of time, in synchronization with either the forward or return stroke of said inner cannula within said outer cannula.

11. A twin irrigating-type electro-cauterizing cannula assembly for use in a hand-supportable powered tissue aspiration instrument having a cannula drive mechanism mounted within a hand-supportable housing having (i) a front end opening extending into a guide tube provided within said hand-supportable housing, (ii) a rear end portion opening for mounting a stationary tubing connector that extends to said guide tube, and (iii) a hollow inner cannula base device driven by said cannula drive mechanism and slidable within said guide tube during instrument operation, wherein said hollow inner base device has a first end portion provided with a second portion of a first leur-lock coupling and a second end portion that slides within the hollow inner portion of said stationary tuning connector, and wherein said front end opening is provided with a first portion of a second leur-lock coupling, said twin irrigating-type electro-cauterizing cannula assembly comprising:
an inner cannula having an inner aspiration aperture formed at the distal portion of said inner cannula and provided with a second portion of said first leur-coupling so that the second portion can be coupled to said first portion of said first leur-coupling provided on said hollow inner cannula base device;
wherein said inner cannula has an inner aspiration aperture formed on said distal end of said inner cannula;
an outer cannula disposed over said inner cannula and having a second portion of said second leur-lock coupling mounted at the proximal end of said outer cannula, so that said second portion can couple to said first portion of said second leur-lock coupling provided at said front end opening of said hand-supportable housing;
wherein said outer cannula has an outer aspiration aperture formed at the distal portion of said outer cannula;
an irrigation channel formed along said outer cannula, and in fluid communication with an irrigation aperture formed at the distal portion of said outer cannula and proximate to said outer aspiration aperture; and
electrodes disposed about said outer aspiration aperture for delivering RF signals aspirated fat tissue being aspirated through said outer and inner aspiration apertures so as to electro-cauterize said aspirated fat tissue; and
wherein said inner aspiration aperture is reciprocated within said outer cannula when said hollow inner cannula base device is reciprocated within said guide tube by said cannula drive mechanism, and therewhile said fluid flows out proximate to said outer aspiration aperture and aspirated back through the outer and inner aspiration apertures down said inner cannula, during the aspiration and electro-cauterization of aspirated fat tissue through said reciprocating inner aspiration aperture.

12. The twin irrigating-type electro-cauterizing cannula assembly of claim 11, wherein said hand-supportable powered tissue aspiration instrument further comprises:
an infusion pump, operably connected to said outer cannula, for delivering controlled amounts of fluid to said irrigation channel, so that said fluid flows out proximate to said outer aspiration aperture and is suctioned back through the outer and inner aspiration apertures, during the aspiration of fat tissue through said reciprocating inner aspiration aperture.

13. The twin irrigating-type electro-cauterizing cannula assembly of claim 12, wherein said hand-supportable powered tissue aspiration instrument further comprises:
a RF signal generating module for generating RF-power signals serving as electro-cauterizing signals about said outer aspiration aperture of said outer cannula.

14. The twin irrigating-type electro-cauterizing cannula assembly of claim 13, wherein said hand-supportable powered tissue aspiration instrument further comprises:
a vacuum pump operably connected to said inner cannula, for aspirating fat tissue from said patient, through said outer and inner aspiration apertures of said twin irrigating electro-cauterizing cannula assembly; and
a controller for controlling said RF signal generating module and said infusion pump to support electro-cauterization and irrigation operations about said inner and outer aspiration apertures, during tissue aspiration operations.

15. A twin electro-cauterizing cannula assembly for use in a hand-supportable powered tissue aspiration instrument having a cannula drive mechanism mounted within a hand-supportable housing having (i) a front end opening extending into a guide tube provided within said hand-supportable housing, (ii) a rear end portion opening for mounting a stationary tubing connector that extends to said guide tube, and (iii) a hollow inner cannula base device driven by said cannula drive mechanism and slidable within said guide tube during instrument operation, wherein said hollow inner base device has a first end portion provided with a second portion of a first leur-lock coupling and a second end portion that slides within the hollow inner portion of said stationary tuning connector, and wherein said front end opening is provided with a first portion of a second leur-lock coupling, said twin electro-cauterizing cannula assembly comprising:
an inner cannula having an inner aspiration aperture formed at the distal portion of said inner cannula and provided with a second portion of said first leur-coupling so that the second portion can be coupled to said first portion of said first leur-coupling provided on said hollow inner cannula base device;
wherein said inner cannula has an inner aspiration aperture formed on said distal end of said inner cannula;
an outer cannula disposed over said inner cannula and having a second portion of said second leur-lock coupling mounted at the proximal end of said outer cannula, so that said second portion can couple to said first portion of said second leur-lock coupling provided at said front end opening of said hand-supportable housing;
wherein said outer cannula has an outer aspiration aperture formed at the distal portion of said outer cannula; and
electrodes disposed about said outer aspiration aperture for delivering RF signals aspirated fat tissue being aspirated through said outer and inner aspiration apertures so as to electro-cauterize said aspirated fat tissue; and
wherein said inner aspiration aperture is reciprocated within said outer cannula when said hollow inner cannula base device is reciprocated within said guide tube by said cannula drive mechanism, during the aspiration and electro-cauterization of aspirated fat tissue through said reciprocating inner aspiration aperture.

16. The twin irrigating-type electro-cauterizing cannula assembly of claim 15, wherein said hand-supportable powered tissue aspiration instrument further comprises:
a RF signal generating module for generating RF-power signals serving as electro-cauterizing signals about said outer aspiration aperture of said outer cannula.

18. The twin irrigating-type electro-cauterizing cannula assembly of claim 16, wherein said hand-supportable powered tissue aspiration instrument further comprises:
a vacuum pump operably connected to said inner cannula, for aspirating fat tissue from said patient, through said outer and inner aspiration apertures of said twin electro-cauterizing cannula assembly; and
a controller for controlling said RF signal generating module to support electro-cauterization operations about said inner and outer aspiration apertures, during tissue aspiration operations.
